(19) United States
(12) Patent Application Publication
(10) Pub. No.: US 2022/0095940 A1
(43) Pub. Date: Mar. 31, 2022

(54) IV DRESSING WITH EMBEDDED SENSORS FOR MEASURING FLUID INFILTRATION AND PHYSIOLOGICAL PARAMETERS

(71) Applicants: BAXTER INTERNATIONAL INC., Deerfield, IL (US); BAXTER HEALTHCARE SA, Glattpark (Opfikon) (CH)

(72) Inventors: Matthew Banet, Deerfield, IL (US); Mark Dhillon, Deerfield, IL (US); Erik Tang, Deerfield, IL (US); Marshal Dhillon, Deerfield, IL (US); James McCanna, Deerfield, IL (US); Chethanya Eleswarpu, Deerfield, IL (US); James P. Martucci, Deerfield, IL (US); Matthew A. Bivans, Deerfield, IL (US); Justin Buckingham, Deerfield, IL (US); Ahren Ceisel, Deerfield, IL (US); Michael Needham, Deerfield, IL (US); Lauren Hayward, Deerfield, IL (US)

(21) Appl. No.: 17/398,268

(22) Filed: Aug. 10, 2021

Related U.S. Application Data
(60) Provisional application No. 63/064,690, filed on Aug. 12, 2020.

(51) Int. Cl. A61B 5/0215; A61B 5/021; A61B 5/00; A61B 5/11

(52) U.S. Cl. CPC A61B 5/02152; A61B 5/02141; A61B 5/02156; A61B 5/6852; A61B 2562/0219; A61B 5/726; A61B 5/7203; A61B 2562/0247; A61B 5/11

(57) ABSTRACT

The invention provides an intravenous (IV) dressing system that helps secure an IV catheter to a patient while simultaneously using embedded peripheral venous pressure (PVP), impedance, temperature, optical, and motion sensors to characterize properties of the IV system (e.g., infiltration, extravasation, occlusion) and the patient's physiological parameters (e.g., heart rate, SpO2, respiration rate, temperature, and blood pressure). Notably, the system converts PVP waveforms into arterial BP values (e.g., systolic and diastolic blood pressure).

### PRIORITY CLAIM AND CROSS-REFERENCE TO RELATED APPLICATIONS

This application claims priority to, and the benefit of, U.S. Provisional Patent App. No. 63/064,690, filed Aug. 12, 2020, entitled IV DRESSING WITH EMBEDDED SENSORS FOR MEASURING FLUID INFILTRATION AND PHYSIOLOGICAL PARAMETERS, the entire contents of which are incorporated by reference herein and relied upon.

### BACKGROUND OF THE INVENTION

### 1. Field of the Invention

The invention described herein relates to systems for drug and fluid delivery, and to systems for monitoring patients in, e.g., hospitals and medical clinics.

### 2. General Background

Unless a term is expressly defined herein using the phrase “herein ‘ ’”, or a similar sentence, there is no intent to limit the meaning of that term beyond its plain or ordinary meaning. To the extent that any term is referred to in this document in a manner consistent with a single meaning, that is done for sake of clarity only; it is not intended that such claim term be limited to that single meaning. Finally, unless a claim element is defined by reciting the word “means” and a function without the recital of any structure, it is not intended that the scope of any claim element be interpreted based on the application of 35 U.S.C. § 112(f).

Proper care of hospitalized patients typically requires: 1) delivery of medications and fluids using intravenous (herein “IV”) catheters and infusion pumps; and 2) measuring vital signs and hemodynamic parameters with patient monitors. Typically, IV catheters are inserted into veins in the patient's hands or arms, and patient monitors are connected to sensors worn on (or inserted in) the patient's body. IV catheters are typically held in place using a large adhesive bandage or dressing, the most common of which has the trade name of “Tegaderm” and is marketed by the 3M Corporation based in Saint Paul, Minn. In addition to its adhesive backing, Tegaderm may include an anti-microbial coating to reduce the occurrence of infection at the IV site. Tegaderm and related IV dressings typically lack any sensors for measuring physiological parameters, such as the ones described above.

IV systems typically use an infusion pump or IV bag to control delivery of fluids. The infusion pump or IV bag are connected through tubing or ‘IV sets’ to the catheter, inserted in the patient's vein. In some cases, the catheter may slip out of the vein and erroneously deliver fluids to surrounding tissue; this instance is referred to herein as “IV infiltration”. Common signs of IV infiltration include inflammation, tightness of the skin, and pain around the site where the catheter is inserted. When left unchecked and untreated, IV infiltration can result in severe pain, infection, compartment syndrome, and even amputation of the affected limb. When the leaked solution from an infiltration is a vesicant drug—one that causes tissue injury, blisters, or severe tissue damage—it is referred to as an ‘extravasation’. Injuries from this type of IV failure can be severe and can lead to the loss of function in an extremity, and if the damage is severe enough, tissue death (also known as necrosis). In still other cases, the catheter's tip can get clogged with a blood clot or medication, thus impeding flow of liquid into the patient's vein; this is referred to herein as “IV occlusion”.

An IV infiltration is a common complication and source of line with IV system; possibly as many as 23% of peripheral IV lines fail due to infiltration (Helm R E, Klausner J D, Klemperer J D, Flint L M, Huang E., “Accepted but unacceptable: peripheral IV catheter failure.”, J Infus. Nurs. 2015; 38(3):189-203). There are many sources of IV infiltration, including clinician error during IV placement, limb movement causing the tip of the catheter to dislodge or poke through the vein well, fragile veins bursting due to high flow rates, and acidic or high osmolarity drug effects on the vein wall. Extravasation, in turn, occurs between 0.1-6% of patients receiving chemotherapy (Al-Benna S, O'Boyle C, Holley J., “Extravasation injuries in adults.”, ISRN Dermatol. 2013; 2013:856541).

Due to the myriad of causes, the incidence of IV infiltration varies by patient population and care setting. IV infiltration has the highest incidence in pediatric and neo-natal populations, especially in the intensive care units serving this demographic. Here, peripheral IVs are common, but smaller vasculature of the patients and commensurate catheter gauges make them more difficult to place and lead to a relatively high occurrence of IV infiltration. Other patient populations, like the elderly or the morbidly obese, are also at a higher risk of IV infiltration due to sources such as fragile veins and difficult placements.

In most hospital settings, patient monitors are used alongside IV systems to measure vital signs and hemodynamic parameters from the patient. Conventional patient monitors typically measure electrocardiogram (herein “ECG”) and impedance pneumography (herein “IP”) waveforms using torso-worn electrodes, from which they calculate heart rate (herein “HR”), heart rate variability (herein “HRV”), and respiration rate (herein “RR”). Most conventional monitors also measure optical signals, called photoplethysmogram (herein “PPG”) waveforms, with sensors that typically clip on the patient's fingers or earlobes. Such sensors can calculate blood oxygen levels (herein “SpO2”) and pulse rate (herein “PR”) from these PPG waveforms. More advanced monitors can also measure blood pressure (herein “BP”), notably systolic (herein “SYS”), diastolic (herein “DIA”), and mean (herein “MAP”) BP. Digital stethoscopes, which can be either portable and body-worn devices, can measure phonocardiogram (herein “PCG) waveforms that indicate heart sounds and murmurs.

BP is a critically important vital sign that can be particularly challenging to measure. The ‘gold standard’ for BP measurement is the arterial line, which is an invasive catheter featuring a transducer that directly measures arterial pressure. The catheter is inserted into an artery (typically the radial, brachial, or femoral artery), and the transducer detects mechanical pressure and coverts it into kinetic energy which can be displayed on the patient monitor. The displayed measurements can include values of SYS, DIA, and MAP, along with a time-dependent pressure waveform. The arterial line, while widely used as a direct beat-to-beat measurement, is highly invasive. It is thus at risk of complications such as infection and can be painful to the patient.

In contrast to arterial lines, an indirect, non-invasive method of detecting BP is a sphygmomanometer, a which is an inflatable cuff that collapses and releases an underlying artery in a controlled way. Sphygmomanometers rely on a manual palpatory method involving inflating a cuff on a patient's upper arm (e.g., bicep) while a clinician palpates the radial artery. The clinician inflates the cuff to a pressure that cause the pulse to disappear; as the cuff is deflated the pressure at which the pulse reappears due to the artery being released is the SYS.

Another manual method using a sphygmomanometer is auscultation, which involves listening to the artery via a stethoscope while a cuff wrapped around the patient's bicep is inflated and then deflated. Similar to the palpatory method, during auscultation the clinician inflates the cuff above the patient's arterial pressure. The clinician then slowly deflates the cuff, which results in the appearance of a ‘Korotkoff sound’ that signals the SYS. Korotkoff sounds are generated as a bolus of blood spurts through the occluded artery when the pressure in the artery rises above the pressure in the cuff. The spurts of blood create turbulence, creating an audible sound. Once the cuff is deflated sufficiently, the Korotkoff sounds disappear, signaling DIA as laminar blood flow through the artery is restored.

Automatic methods using cuff-based systems similar to the sphygmomanometer are also widely used to measure BP. One of the most common methods is oscillometry. Here, the cuff features a pressure transducer that detects time-dependent changes in the cuff pressure. During a measurement, and with each arterial pulse, blood flow causes the volume of the patient's arm to change slightly, thereby creating a small pressure pulse in the cuff that the pressure transducer detects. As the cuff inflates, the device can detect when the blood flow is stopped by the absence of the pulses. The device then slowly deflates the cuff, at which point the appearance of small pressure pulses indicate SYS, and the subsequent disappearance of those pulses indicate DIA and the return of laminar blood flow.

While the methods using auscultation and oscillometry are non-invasive, there still is a varying level of tolerance among patients due to the cuff's uncomfortable nature. Additionally, these methods are intermittent and have limited value for situations in which continuous blood pressure measurement would be clinically useful, such as vasopressor titration.

Recent advances have also led to non-invasive BP measurements that are also continuous. Such methods involve using the volume clamp technique, arterial applanation tonometry, optical sensors, and multi-sensor techniques that measure ‘systolic time intervals’ and then use algorithms to convert these into BP values.

The volume clamp technique, such as that used by the ‘Clearsight’ (from Edwards Scientific, based in Irvine, Calif.), features a finger cuff and optical sensor that includes a light source and photodiode. The finger cuff is inflated to maintain a consistent diameter of the artery in a finger, which is then measured by the optical sensor. The finger cuff adjusts the pressure to maintain the artery's diameter. These adjustments can be used to calculate a pressure curve that corresponds to SYS and DIA.

Arterial applanation tonometry involves placing a pressure sensor over an artery (typically the radial artery) that is disposed over bone. During a measurement, pressure applied by the device causes the sensor to press against the artery. The pressure sensor measures the pressure needed to flatten the artery wall, leading to measurements of SYS and DIA.

In yet another technique that is both non-invasive and continuous, sensors that simultaneously measure PPG and ECG waveforms can yield an estimate of BP by measuring systolic time intervals, i.e., the duration of time it takes for a signal to propagate between two points in the patient. A specific technique, called pulse transit time (herein “PTT”), is the time separating a heartbeat-induced pulse in a PPG or PCG waveform (typically measured from the chest or arm) and a pulse measured at a different location on the body (typically a PPG waveform measured at the finger). Pulse arrival time (herein “PAT”) uses a similar concept, except that it measures the time separating an ECG R-wave (typically measured from the chest) and a pulse in a PPG waveform (typically measured at the finger). PAT differs from PTT in that includes the pre-ejection period (herein “PEP”) and isovolumic contraction time (herein “ICT”). Both PTT and PAT inversely relate to BP, and most measurements based these techniques are calibrated with a cuff-based system, and typically an automated system based on oscillometry, to yield absolute measurements of SYS and DIA. The “ViSi” system (from Sotera Wireless based in San Diego, Calif.) is a commercially available BP-measuring device based on PAT.

Some patient monitors are entirely body-worn. These typically take the shape of patches that measure ECG, HR, HRV and, in some cases, RR. Such patches can also include accelerometers that measure motion (herein “ACC”) waveforms. Algorithms can determine the patient's posture, degree of motion, falls, and other related parameters from the ACC waveforms. Patients typically wear these types of patches in the hospital; alternatively they are used for ambulatory and home use. The patches are typically worn for relatively short periods of time (e.g., from a few days to several weeks). They are typically wireless, and usually include technologies such as Bluetooth® transceivers to transmit information over a short range to a secondary ‘gateway’ device, which typically includes a cellular or Wi-Fi radio to transmit the information to a cloud-based system.

Even more complex patient monitors measure parameters such as stroke volume (herein “SV”), cardiac output (herein “CO”), and cardiac wedge pressure using an invasive sensor called a Swan-Ganz or pulmonary-artery catheter. To make a measurement, these sensors are positioned in the patient's left heart, where they are ‘wedged’ into a small pulmonary blood vessel using a balloon catheter. As an alternative to this highly invasive measurement, patient monitors can use non-invasive techniques such as bio-impedance and bio-reactance to measure similar parameters. These methods deploy body-worn electrodes on any body part (and typically deployed on the patient's chest, legs, and/or neck) to measure bio-impedance plethysmogram (herein “IMP”) and/or bio-reactance (herein “BR”) waveforms. Analysis of IMP and BR waveforms yields SV, CO, and thoracic impedance, which is a proxy for fluids in the patient's chest (herein “FLUIDS”). Notably, IMP and BR waveforms generally have similar shapes and are sensed using similar measurement techniques and are thus used interchangeably herein.

Devices that measure BP, and less commonly SV, CO, and FLUIDS, can yield metrics that allow clinicians to estimate a patient's blood volume, fluid responsiveness, and, in some cases, related metrics such as central venous pressure (herein “CVP”). Taken collectively, these parameters can diagnose certain medical conditions and guide resuscitation efforts. But the highly invasive nature of Swan-Ganz and pulmonary-artery catheters can be disadvantageous and comes with a high risk of infection. Additionally, CVP measurements may be slower to change in response to certain acute conditions, such as when the circulatory system attempts to compensate for blood volume disequilibrium (particularly hypovolemia) by protecting blood volume levels in the central circulatory system at the expense of the periphery. For example, constriction in peripheral blood vessels may reduce the effect of fluid loss on the central system, thereby temporarily masking blood loss in conventional CVP measurements. Such masking can lead to delayed recognition and treatment of patient conditions, thereby worsening outcomes.

To address these and other shortcomings, a measurement technique called peripheral intravenous waveform analysis (herein “PIVA”) has been developed, as described in U.S. patent application Ser. No. 14/853,504 (filed Sep. 14, 2015 and published as U.S. Patent Publication No. 2016/0073959) and PCT Application No. PCT/US16/16420 (filed Feb. 3, 2016, and published as WO 2016/126856), the contents of which are incorporated herein by reference. These documents describe sensors featuring pressure transducers that receive signals from in-dwelling catheters inserted in a patient's venous system, and connect through cables to remote electronics that process signals generated therefrom (herein “PIVA sensor”). PIVA sensors measure time-dependent waveforms indicating peripheral venous pressure (herein “PVP”) using existing IV lines, which typically include IV tubing attached to a saline drip or infusion pump. PVP waveforms can be filtered to show relatively high-frequency signal components (herein “PVP-AC” waveforms) and low-frequency signal components (herein “PVP-DC” waveforms). The ‘AC’ term is normally used to describe alternating current but is used herein to indicate a signal component that changes rapidly in time. Likewise, low-frequency components of the PVP waveforms are relatively stable and unvarying over time and are thus indicated by the term ‘DC’, which is normally used to describe direct current and corresponding signals that do not rapidly change with time. Measurements made with PIVA sensors typically feature a mathematical transformation of the PVP waveforms (and typically PVP-AC waveforms) into the frequency domain, performed with a remote computer, using a methodology called fast Fourier Transform (herein “FFT”). Analysis of a frequency-domain spectrum generated with an FFT can yield a RR frequency (herein “F0”) and a HR frequency (herein “F1”) indicating, respectively, the patient's HR and RR. A more detailed analysis of F0 and F1, e.g., use of a computer algorithm to determine the amplitude of these peaks or, alternatively, integrate an area underneath the curve centered around the maximum peak amplitude, determines the ‘energy’ of these features. Further processing of these energies yields an indication of a patient's blood volume status. Such measurements have been described, for example, in the following references, the contents of which are herein incorporated by reference: 1) Hocking et al., “Peripheral venous waveform analysis for detecting hemorrhage and iatrogenic volume overload in a porcine model.”, Shock. 2016 October; 46(4):447-52; 2) Sileshi et al., “Peripheral venous waveform analysis for detecting early hemorrhage: a pilot study.”, Intensive Care Med. 2015 June; 41(6):1147-8; 3) Miles et al., “Peripheral intravenous volume analysis (PIVA) for quantitating volume overload in patients hospitalized with acute decompensated heart failure—a pilot study.”, J Card Fail. 2018 August; 24(8):525-532; and 4) Hocking et al., “Peripheral i.v. analysis (PIVA) of venous waveforms for volume assessment in patients undergoing haemodialysis.”, Br J Anaesth. 2017 Dec. 1; 119(6):1135-1140.

Unfortunately, during typical measurements with PIVA sensors, PVP waveforms induced by HR and RR events (typically 5-20 mmHg) are much weaker than their arterial pressure counterparts (typically 60-150 mmHg). This means magnitudes of corresponding signals in time-dependent PVP waveforms measured by conventional pressure transducers are often very weak (e.g., typically 5-50 □V). Additionally, PVP waveforms are typically amplified, conditioned, digitized, and ultimately processed with electronic systems located remotely from the patient. Thus, prior to these steps, analog versions of the waveforms travel through cables that can attenuate them and add noise (due, e.g., to motion). And in some cases, PVP waveforms simply lack signatures corresponding to F0 and F1. Or peaks of one primary frequency are obscured by ‘harmonics’ (i.e., integer multiple of a given frequency) of the other primary frequency. This can make it difficult or impossible for an automated medical device to accurately determine F0 and F1, and the energy associated with these features.

### SUMMARY OF THE INVENTION

In view of the foregoing, it would be beneficial to provide an IV dressing system (herein “IVDS”) that provides the functions of a Tegaderm-like dressing—i.e., a bandage-like component that secures an IV to a patient—while simultaneously characterizing properties of the IV system (e.g., infiltration, extravasation, occlusion) and the patient's physiological parameters (e.g., HR, HRV, SpO2, RR, TEMP, and BP). In particular, it would be beneficial if the IVDS could measure PVP signals—which result from the patient's venous system—and convert them into arterial BP values (e.g., SYS, MAP, DIA).

To make such measurements, the IVDS would improve on a conventional PIVA sensor so that it overcomes historical problems related to weak, noisy PVP waveforms, and also incorporate a set of sensors that simultaneously measures signals related to the IV system and patient. Such as system could improve how patients are monitored in hospitals and medical clinics. To cure these and other deficiencies, the IVDS features embedded impedance, temperature, and motion sensors, and an augmented, improved PVP sensor featuring a circuit board located in close proximity to an in-dwelling venous catheter that amplifies, filters, and digitizes PVP waveforms immediately after a pressure sensor detects them (e.g., directly on the patient's body).

Additionally, according to the invention, measurements from the PVP sensor can be coupled with independent measurements of hemodynamic parameters, e.g., SV, CO, and FLUIDS (which can be made with the patch sensor or a comparable patient monitor) to yield an improved understanding of the patient's fluid status.

The IVDS described herein is designed to work with a conventional IV system and features a dressing component that is flexible and adhesive; it connects the in-dwelling catheter to the patient. The IV system, dressing, and catheter are all standard equipment used in the hospital. The dressing typically includes at least four embedded electrodes, typically made from a hydrogel-based material, that perform an impedance measurement that senses the accumulation of fluid that, during some IV treatments, is erroneously deposited outside of the patient's vein and accumulates in surrounding tissue. Additionally, the dressing may include a temperature sensor and optical sensor that detect, respectively, temperature and optical absorption changes that relate to the accumulating fluid. A motion sensor (e.g., an accelerometer and/or gyroscope) within the IVDS characterizes the patient's motion to eliminate false negative and positive readings while simultaneously characterizing the patient's posture (e.g., standing, sitting, lying supine) and activity level (e.g., walking, sleeping, falling). The catheter includes a housing, worn close to or on the patient's body, and typically on their arm or hand, that encloses a PVP-conditioning circuit board featuring complex circuitry that amplifies, filters, and digitizes analog PVP waveforms. The circuit board may also include components for processing and storing the digitized signals, and wirelessly transmitting information (e.g., a Bluetooth® transmitter). In this way, the circuit board can integrate with a remote processor (e.g., server, gateway, tablet, smartphone, computer, infusion pump, or some combination thereof) that can display information from the IVDS, generate alarms and alerts related to the patient's physiology and IV system, and collectively analyze complementary information from other patient-worn devices, e.g., a patch sensor.

The IVDS described herein simplifies the processes of securing an IV to and patient, characterizing the performance of the IV, and measuring traditional measurements of vital signs and hemodynamic parameters, which can involve multiple devices and can take several minutes to accomplish. The remote processor—which wirelessly couples with IVDS—can additionally integrate with existing hospital infrastructure and notification systems, such as a hospital's electronic medical records (herein “EMR”) system. Such a system can alarm and alert caregivers to changes in a patient's condition, thereby allowing them to intervene.

The IVDS typically features a low-cost disposable system that includes electrodes on its bottom surface that secure it to the patient's body without requiring bothersome cables. The disposable system typically connects to a reusable system that features relatively expensive electronic components, such as a printed circuit board (herein “PCB) featuring a microprocessor, memory, sensing electronics, a wireless transmitter, and a rechargeable Li-ion battery. In embodiments, the disposable component connects to the reusable component by means of magnets, thus allowing one component to easily snap back into proper with the other if it is removed. The entire IVDS—both reusable and disposable components—is typically lightweight, weighing about 20 grams. The Li:ion battery can be recharged with a conventional cable (e.g., one that connects to a remote infusion pump or display module) or using a wireless mechanism.

Given the above, in one aspect the invention provides a system for determining an arterial BP value (i.e., SYS, DIA, and MAP) from a patient. The system features: 1) a catheter that inserts into the patient's venous system; 2) a pressure sensor connected to the catheter that measures physiological signals indicating a pressure in the patient's venous system; and 3) a processing system configured to: i) receive the physiological signals from the pressure sensor; and ii) process the physiological signals with an algorithm to determine the arterial BP value.

In embodiments, the processing system is further configured to operate an algorithm that filters out respiratory components from the physiological signals to determine the arterial BP value. For example, to perform this filtering, the algorithm may operate a bandpass filter or use a filtering approach based on wavelets (e.g., a continuous wavelet transform (herein “CWT”), a discrete wavelet transform (herein “DWT”), or an adaptive filter that uses parameters determined from another sensor, e.g., a patch sensor) to filter out the respiratory components.

In other embodiments, the IVDS includes an enclosure that attaches directly to the patient covers the processing system, which is typically a circuit board that features a microprocessor. The processing system can further include a motion-detecting sensor, such as an accelerometer (and typically a 3-axis accelerometer) or gyroscope. In embodiments, the processing system is further configured to receive signals from the motion-detecting sensor and process them to determine the patient's degree of motion. The processing system then collectively processes this parameter and the patient's physiological signals to determine BP. In other embodiments, the processing system is further configured to process signals from the motion-detecting sensor to determine a relative height associated with a body part (e.g., an arm, wrist, or hand) associated with the patient. Here, for example, the signals may be those detected along one axis of the 3-axis accelerometer. The processing system can then collectively process the relative height associated with the body part and the physiological signals to determine the arterial BP value.

In other embodiments, the system interfaces with an external calibration source (e.g., a blood pressure cuff or arterial catheter) that measures BP with an established, conventional technology. Here, the processing system is further configured to receive a calibration BP value from the external source, and then process the calibration BP value with the physiological signals to determine the arterial BP value. In related embodiments, the processing system is further configured to determine and then process a patient-specific relationship between venous BP and arterial BP, along with the calibration BP value and the physiological signals, to determine the arterial BP value. Here, the patient-specific relationship between venous BP and arterial BP can be derived from the physiological signals that the pressure sensor measures, or from biometric information corresponding to the patient (e.g., the patient's gender, age, weight, height, or BMI).

In other embodiments, the system additionally includes a wireless transceiver (e.g., a Bluetooth®, Wi-Fi, or a cellular transceiver) that wirelessly receives the calibration BP value from the external source, which in turn includes a paired wireless transceiver. Additionally, the wireless transceiver can also wirelessly transmit the arterial BP value to an external display system (e.g., an infusion pump, a remote display, a computer, a mobile phone, or a medical records system).

In another aspect, the invention provides a system for determining when a liquid solution (e.g., saline or medication mixed with a liquid like saline) provided by an intravenous delivery system is delivered outside of a vein within a patient. The system features: 1) a catheter that inserts into the vein; 2) a pressure sensor connected to the catheter that measures pressure signals indicating a pressure within the vein; 3) an impedance-measuring system that measures impedance signals indicating an electrical impedance of tissue proximal to the vein; and 4) a processing system configured to: i) receive the pressure signals from the pressure sensor; ii) receive the impedance signals from the impedance-measuring system; and iii) collectively process the pressure signals and the impedance signals with an algorithm to determine when the liquid solution provided by the intravenous delivery system is delivered outside of the vein.

In embodiments, the algorithm is configured to evaluate time-dependent changes in the pressure signals to determine when the liquid solution provided by the intravenous delivery system is delivered outside of the vein. For example, the time-dependent changes may indicate that the pressure increases or decreases (typically in a rapid manner) within the vein. Or they may be the sudden presence or absence of short-term pressure pulses induced by the patient's heart, or the presence or absence of long-term pressure pulses induced by the intravenous delivery system.

In related embodiments, the algorithm is further configured to evaluate time-dependent changes in the impedance signals to determine when the liquid solution provided by the intravenous delivery system is delivered outside of the vein. For example, the time-dependent changes in the impedance signals may be an increase or decrease in electrical impedance measured from tissue proximal to the vein. In related embodiments, the processing system is further configured to evaluate the electrical conductivity of the liquid solution provided by an intravenous delivery system. This is because a liquid with relatively high electrical conductivity (compared to the patient's tissue) will cause the measured impedance to decrease, whereas as a liquid with relatively low conductivity will cause it to increase.

In other embodiments, the system includes a flexible substrate (e.g., an adhesive pad or bandage) that secures the catheter to the patient. The flexible substrate can include a set of electrodes (e.g., those made from a hydrogel material). In embodiments, each electrode in the set of electrodes is in electrical contact with the impedance-measuring system, and at least one electrode is configured to inject electrical current into the tissue proximal to the vein, while at least one other electrode in the set of electrodes is configured to measure a signal induced by the electrical current. For example, in embodiments, at least two electrodes in the set of electrodes are configured to measure a voltage change induced by the electrical current.

In embodiments, the impedance-measuring system is comprised of a collection of discrete circuit components. Alternatively, it may be just a single integrated circuit.

In other embodiments, the system further includes a temperature sensor that measures time-dependent temperature signals indicating temperature in the tissue proximal to the vein. Typically, IV infiltration is characterized by a rapid drop in temperature, as the infiltrating fluid is typically at room temperature (e.g., around 70° F.) whereas the human body features a relatively higher temperature (e.g., around 98-99° F.). In some cases, however, an increase in temperature indicates IV infiltration. In either case, in this embodiment, the processing system is further configured to: 1) receive the temperature signals from the temperature sensor; and ii) collectively process the temperature signals, along with pressure signals and the impedance signals, with an algorithm to determine when the liquid solution provided by the intravenous delivery system is delivered outside of the vein.

In other embodiments, the processing system is further configured to process the pressure signals or the impedance signals, or some combination thereof, to determine at least one physiological parameter (e.g., HR, RR, or FLUIDS) corresponding to the patient.

In embodiments, the processing system additionally processes the signal components related to the patient's HR and RR to determine a physiological parameter (e.g., wedge pressure, central venous pressure, blood volume, fluid volume, and pulmonary arterial pressure) indicating the patient's fluid status.

In embodiments, the processing system transforms the signals into the frequency domain to generate a frequency-domain signal prior to determining the physiological parameter. The method for the transform is typically an FFT, CWT, or a DWT.

In embodiments, the low-pass filter typically separates out from the amplified signal a signal component containing HR and RR components. The low-pass filter typically includes circuit components that generate a filter cutoff of between 10 and 30 Hz. In other embodiments, the circuit system additionally includes a high-pass filter that receives the twice-amplified signals and, in response, generates a twice-filtered signal. In this case, the high-pass filter typically includes circuit components that generate a filter cutoff of between 0.01 and 1 Hz.

In embodiments, the circuit system additionally includes a secondary low-pass filter that receives the twice-amplified signals and, in response, generates a thrice-filtered signal. In this case, the secondary low-pass filter typically includes circuit components that generate a filter cutoff of between 10 and 30 Hz.

In other embodiments, the system additionally includes a flash memory system that stores a digital representation of the twice-amplified signal or a signal derived therefrom.

In embodiments, the bio-impedance system can be replaced by a bio-reactance sensing system. In other embodiments, the physiological parameters measured by the system are selected from a group including BP, SpO2, SV, stroke index, CO, cardiac index, thoracic impedance, FLUIDS, inter-cellular fluids, and extra-cellular fluids. In other embodiments, the second set of parameters are selected from a group including F0, F1, energies associated with F0 and F1, mathematical combinations of F0 and F1, and parameters determined from these.

The processing system can operate a linear mathematical model to collectively process the signals described above. Alternatively, it can operate an algorithm based on artificial intelligence to collectively process the first and second sets of parameters.

In another aspect, the invention provides a system for monitoring a physiological parameter from a patient and determining when a liquid solution provided by a vein-inserted catheter is delivered outside of the vein. The system features a flexible substrate (e.g., a bandage-type component) secures the catheter to the patient and includes at least one sensor. The sensor measures signals that indicate the physiological parameter and determine when the liquid solution is delivered outside the vein. The system also includes a processing system that: i) receives the signals from the sensor; ii) processes the signals with a first algorithm to determine the physiological parameter; and iii) processes the signals with a second algorithm to determine when the liquid solution provided by the catheter is delivered outside of the vein.

In embodiments, the sensor is at least one electrode (e.g., an electrode that features a hydrogel component). More typically, the sensor includes at least four electrodes, and the system additionally includes an electrical impedance circuit that electrically connects to each of the four electrodes. The electrical impedance circuit can inject electrical current into a first set of electrodes, and measure bio-electric signals from a second set of electrodes. During a measurement, the circuit process the bio-electric signals from the second set of electrodes to generate a time-dependent IMP waveform. The processing system then receives the time-dependent IMP waveform, and the first algorithm it operates processes the time-dependent IMP waveform to determine a value of HR, RR, or fluids. The second algorithm it operates additionally processes the time-dependent IMP waveform to determine when the liquid solution provided by the catheter is delivered outside of the vein.

In another embodiment, the sensor is a temperature sensor (e.g., a thermistor, thermocouple, resistance temperature detector, thermometer, optical sensor, and thermal flow sensor). Here, the system further includes a temperature-measuring circuit that electrically connects to the temperature sensor. During a measurement, the temperature-measuring circuit processes the signals from the temperature sensor to generate a time-dependent temperature waveform. The processing system then receives the time-dependent IMP waveform, and the first algorithm it operates processes it to determine a value of skin temperature or core temperature. The second algorithm it operates additionally processes the time-dependent temperature waveform to determine when the liquid solution provided by the catheter is delivered outside of the vein.

In other embodiments, the system includes a motion sensor (e.g., an accelerometer or gyroscope), and the motion sensor generates a time-dependent motion waveform (e.g., along one of its three axes). The processing system can receive the time-dependent motion waveform and analyze it and the sensor-generated signals to determine the physiological parameter. Additionally, the processing system is further configured to receive the time-dependent motion waveform and analyze it and the sensor-generated signals to determine when the liquid solution provided by the catheter is delivered outside of the vein.

In light of the disclosure herein, disclosure herein, and without limiting the scope of the invention in any way, in a first aspect of the present disclosure, which may be combined with any other aspect listed herein unless specified otherwise, a system for determining an arterial blood pressure value from a patient includes a catheter, a pressure sensor, and a processing system. The catheter is configured to insert into the patient's venous system. The pressure sensor is connected to the catheter and configured to measure physiological signals indicating a pressure in the patient's venous system. The processing system is configured to: i) receive the physiological signals from the pressure sensor; and ii) process the physiological signals with an algorithm to determine the arterial blood pressure value.

In a second aspect of the present disclosure, which may be combined with any other aspect listed herein unless specified otherwise, the processing system is further configured to operate an algorithm that filters out respiratory components from the physiological signals to determine the arterial blood pressure value.

In a third aspect of the present disclosure, which may be combined with any other aspect listed herein unless specified otherwise, the algorithm is further configured to operate a bandpass filter to filter out respiratory components from the physiological signals.

In a fourth aspect of the present disclosure, which may be combined with any other aspect listed herein unless specified otherwise, the algorithm is further configured to operate a filter based on wavelets to filter out respiratory components from the physiological signals.

In a fifth aspect of the present disclosure, which may be combined with any other aspect listed herein unless specified otherwise, the processing system is enclosed by an enclosure that is configured to attach directly to the patient.

In a sixth aspect of the present disclosure, which may be combined with any other aspect listed herein unless specified otherwise, the processing system further comprises a motion-detecting sensor.

In a seventh aspect of the present disclosure, which may be combined with any other aspect listed herein unless specified otherwise, the motion-detecting sensor is one of an accelerometer and a gyroscope.

In an eighth aspect of the present disclosure, which may be combined with any other aspect listed herein unless specified otherwise, the processing system is further configured to receive signals from the motion-detecting sensor and process them to determine the patient's degree of motion.

In a ninth aspect of the present disclosure, which may be combined with any other aspect listed herein unless specified otherwise, the processing system is further configured to collectively process the patient's degree of motion and the physiological signals to determine the arterial blood pressure value.

In a tenth aspect of the present disclosure, which may be combined with any other aspect listed herein unless specified otherwise, the processing system is further configured to receive signals from the motion-detecting sensor and process them to determine a relative height associated with a body part associated with the patient.

In an eleventh aspect of the present disclosure, which may be combined with any other aspect listed herein unless specified otherwise, the body part is the patient's arm.

In a twelfth aspect of the present disclosure, which may be combined with any other aspect listed herein unless specified otherwise, the processing system is further configured to collectively process the relative height associated with the body part associated with the patient and the physiological signals to determine the arterial blood pressure value.

In a thirteenth aspect of the present disclosure, which may be combined with any other aspect listed herein unless specified otherwise, the processing system is further configured to receive a calibration blood pressure value from an external source.

In a fourteenth aspect of the present disclosure, which may be combined with any other aspect listed herein unless specified otherwise, the processing system is further configured to process the calibration blood pressure value with the physiological signals to determine the arterial blood pressure value.

In a fifteenth aspect of the present disclosure, which may be combined with any other aspect listed herein unless specified otherwise, the external source is one of a blood pressure cuff and an arterial catheter.

In a sixteenth aspect of the present disclosure, which may be combined with any other aspect listed herein unless specified otherwise, the processing system is further configured to process a patient-specific relationship between venous blood pressure and arterial blood pressure, along with the calibration blood pressure value and the physiological signals, to determine the arterial blood pressure value.

In a seventeenth aspect of the present disclosure, which may be combined with any other aspect listed herein unless specified otherwise, the processing system is further configured to process the physiological signals to determine the patient-specific relationship between venous blood pressure and arterial blood pressure.

In an eighteenth aspect of the present disclosure, which may be combined with any other aspect listed herein unless specified otherwise, the processing system is further configured to process biometric information corresponding to the patient to determine the patient-specific relationship between venous blood pressure and arterial blood pressure.

In a nineteenth aspect of the present disclosure, which may be combined with any other aspect listed herein unless specified otherwise, the biometric information includes at least one of the patient's gender, age, weight, height, and BMI.

In a twentieth aspect of the present disclosure, which may be combined with any other aspect listed herein unless specified otherwise, the system further includes a wireless transceiver configured to wirelessly receive the calibration blood pressure value from the external source.

In a twenty-first aspect of the present disclosure, which may be combined with any other aspect listed herein unless specified otherwise, the wireless transceiver is one of a Bluetooth®, Wi-Fi, or a cellular transceiver.

In a twenty-second aspect of the present disclosure, which may be combined with any other aspect listed herein unless specified otherwise, the system further includes a wireless transceiver configured to wirelessly transmit the arterial blood pressure value to an external display system.

In a twenty-third aspect of the present disclosure, which may be combined with any other aspect listed herein unless specified otherwise, the external display system is one of an infusion pump, a remote display, a computer, a mobile phone, or a medical records system.

In a twenty-fourth aspect of the present disclosure, which may be combined with any other aspect listed herein unless specified otherwise, a system for determining an arterial blood pressure value from a patient includes a catheter, a pressure sensor, a motion sensor, and a processing system. The catheter is configured to insert into the patient's venous system. The pressure sensor is connected to the catheter and configured to measure physiological signals indicating a pressure in the patient's venous system. The motion sensor is configured to measure motion signals. The processing system is configured to: i) receive the physiological signals from the pressure sensor; ii) receive the motion signals from the motion sensor; iii) process the motion signals by comparing them to a pre-determined threshold value to determine when the patient has a relatively low degree of motion; and iv) process the physiological signals to determine the arterial blood pressure value.

In a twenty-fifth aspect of the present disclosure, which may be combined with any other aspect listed herein unless specified otherwise, a system for determining an arterial blood pressure value from a patient includes a catheter, a pressure sensor, a motion sensor, and a processing system. The catheter is configured to insert into the patient's venous system. The pressure sensor is connected to the catheter and configured to measure physiological signals indicating a pressure in the patient's venous system. The motion sensor is configured to measure motion signals. The processing system is configured to: i) receive the physiological signals from the pressure sensor; ii) receive the motion signals from the motion sensor; iii) process the motion signals to determine a relative height between a body part associated with the patient and an infusion system; and iv) process the physiological signals and the relative height to determine the arterial blood pressure value.

In a twenty-sixth aspect of the present disclosure, which may be combined with any other aspect listed herein unless specified otherwise, a system for determining when a liquid solution provided by an intravenous delivery system is delivered outside of a vein within a patient, includes a catheter, a pressure sensor, an impedance-measuring system, and a processing system. The catheter is configured to insert into the vein. The pressure sensor is connected to the catheter and configured to measure pressure signals indicating a pressure within the vein. The impedance-measuring system is configured to measure impedance signals indicating an electrical impedance of tissue proximal to the vein. The processing system is configured to: i) receive the pressure signals from the pressure sensor; ii) receive the impedance signals from the impedance-measuring system; and iii) collectively process the pressure signals and the impedance signals with an algorithm to determine when the liquid solution provided by the intravenous delivery system is delivered outside of the vein.

In a twenty-seventh aspect of the present disclosure, which may be combined with any other aspect listed herein unless specified otherwise, the algorithm is configured to evaluate time-dependent changes in the pressure signals to determine when the liquid solution provided by the intravenous delivery system is delivered outside of the vein.

In a twenty-eighth aspect of the present disclosure, which may be combined with any other aspect listed herein unless specified otherwise, the time-dependent changes in the pressure signals are one of an increase and decrease in pressure within the vein.

In a twenty-ninth aspect of the present disclosure, which may be combined with any other aspect listed herein unless specified otherwise, the time-dependent changes in the pressure signals are one of the presence and absence of pressure pulses induced by the patient's heart.

In a thirtieth aspect of the present disclosure, which may be combined with any other aspect listed herein unless specified otherwise, the time-dependent changes in the pressure signals are one of the presence or absence of pressure pulses induced by the intravenous delivery system.

In a thirty-first aspect of the present disclosure, which may be combined with any other aspect listed herein unless specified otherwise, the algorithm is further configured to evaluate time-dependent changes in the impedance signals to determine when the liquid solution provided by the intravenous delivery system is delivered outside of the vein.

In a thirty-second aspect of the present disclosure, which may be combined with any other aspect listed herein unless specified otherwise, the time-dependent changes in the impedance signals are one of an increase and decrease in electrical impedance from tissue proximal to the vein.

In a thirty-third aspect of the present disclosure, which may be combined with any other aspect listed herein unless specified otherwise, the processing system is further configured to evaluate the electrical conductivity of the liquid solution provided by an intravenous delivery system.

In a thirty-fourth aspect of the present disclosure, which may be combined with any other aspect listed herein unless specified otherwise, the system further includes a flexible substrate configured to secure the catheter to the patient.

In a thirty-fifth aspect of the present disclosure, which may be combined with any other aspect listed herein unless specified otherwise, the flexible substrate comprises a set of electrodes.

In a thirty-sixth aspect of the present disclosure, which may be combined with any other aspect listed herein unless specified otherwise, each electrode in the set of electrodes comprises a hydrogel material.

In a thirty-seventh aspect of the present disclosure, which may be combined with any other aspect listed herein unless specified otherwise, each electrode in the set of electrodes is in electrical contact with the impedance-measuring system.

In a thirty-eighth aspect of the present disclosure, which may be combined with any other aspect listed herein unless specified otherwise, at least one electrode in the set of electrodes is configured to inject electrical current into the tissue proximal to the vein.

In a thirty-ninth aspect of the present disclosure, which may be combined with any other aspect listed herein unless specified otherwise, at least one electrode in the set of electrodes is configured to measure a signal induced by the electrical current.

In a fortieth aspect of the present disclosure, which may be combined with any other aspect listed herein unless specified otherwise, at least two electrodes in the set of electrodes are configured to measure a voltage change induced by the electrical current.

In a forty-first aspect of the present disclosure, which may be combined with any other aspect listed herein unless specified otherwise, the impedance-measuring system is comprised of a collection of discrete circuit components.

In a forty-second aspect of the present disclosure, which may be combined with any other aspect listed herein unless specified otherwise, the impedance measuring system is comprised of a single integrated circuit.

In a forty-third aspect of the present disclosure, which may be combined with any other aspect listed herein unless specified otherwise, the system further includes a temperature sensor configured to measure time-dependent temperature signals indicating temperature in the tissue proximal to the vein.

In a forty-fourth aspect of the present disclosure, which may be combined with any other aspect listed herein unless specified otherwise, the time-dependent temperature signals are one of an increase and decrease in temperature proximal to the vein.

In a forty-fifth aspect of the present disclosure, which may be combined with any other aspect listed herein unless specified otherwise, the processing system is further configured to: 1) receive the temperature signals from the temperature sensor; and ii) collectively process the temperature signals, along with pressure signals and the impedance signals, with an algorithm to determine when the liquid solution provided by the intravenous delivery system is delivered outside of the vein.

In a forty-sixth aspect of the present disclosure, which may be combined with any other aspect listed herein unless specified otherwise, the processing system is further configured to process the pressure signals to determine at least one physiological parameter corresponding to the patient.

In a forty-seventh aspect of the present disclosure, which may be combined with any other aspect listed herein unless specified otherwise, the physiological parameter is one of heart rate and respiration rate.

In a forty-eighth aspect of the present disclosure, which may be combined with any other aspect listed herein unless specified otherwise, the processing system is further configured to process the impedance signals to determine at least one physiological parameter corresponding to the patient.

In a forty-ninth aspect of the present disclosure, which may be combined with any other aspect listed herein unless specified otherwise, the physiological parameter is one of heart rate and respiration rate.

In a fiftieth aspect of the present disclosure, which may be combined with any other aspect listed herein unless specified otherwise, a system for determining when a liquid solution provided by an intravenous delivery system is delivered outside of a vein within a patient, includes a catheter, a pressure sensor, an impedance-measuring system, a temperature-measuring system and a processing system. The catheter is configured to insert into the vein. The pressure sensor is connected to the catheter and configured to measure pressure signals indicating a pressure within the vein. The impedance-measuring system is configured to measure impedance signals indicating an electrical impedance of tissue proximal to the vein. The temperature-measuring system is configured to measure temperature signals indicating a temperature of tissue proximal to the vein. The processing system is configured to: i) receive the pressure signals from the pressure sensor; ii) receive the impedance signals from the impedance-measuring system; iii) receive the temperature signals from the temperature sensor; and iii) collectively process the pressure signals, impedance signals, and temperature signals with an algorithm to determine when the liquid solution provided by the intravenous delivery system is delivered outside of the vein.

In a fifty-first aspect of the present disclosure, which may be combined with any other aspect listed herein unless specified otherwise, a system for determining a physiological parameter from a patient and when a liquid solution provided by an intravenous delivery system is delivered outside of a vein within the patient, includes a catheter, a pressure sensor, an impedance-measuring system, and a processing system. The catheter is configured to insert into the vein. The pressure sensor is connected to the catheter and configured to measure pressure signals indicating a pressure within the vein. The impedance-measuring system is configured to measure impedance signals indicating an electrical impedance of tissue proximal to the vein. The processing system is configured to: i) receive the pressure signals from the pressure sensor; ii) receive the impedance signals from the impedance-measuring system; iii) collectively process the pressure signals and the impedance signals with an algorithm to determine when the liquid solution provided by the intravenous delivery system is delivered outside of the vein; and iv) process at least one of the pressure signals and the impedance signals to determine the physiological parameter from the patient.

In a fifty-second aspect of the present disclosure, which may be combined with any other aspect listed herein unless specified otherwise, a system for monitoring a physiological parameter from a patient and determining when a liquid solution provided by a catheter configured to insert in a vein within the patient is delivered outside of the vein, includes a flexible substrate, a sensor, and a processing system. The flexible substrate includes at least one sensor and configured to secure the catheter to the patient. The sensor is configured to measure signals that indicate the physiological parameter and determine when the liquid solution is delivered outside the vein. The processing system is configured to: i) receive the signals from the sensor; ii) process the signals with a first algorithm to determine the physiological parameter; and iii) process the signals with a second algorithm to determine when the liquid solution provided by the catheter is delivered outside of the vein.

In a fifty-third aspect of the present disclosure, which may be combined with any other aspect listed herein unless specified otherwise, the sensor is at least one electrode.

In a fifty-fourth aspect of the present disclosure, which may be combined with any other aspect listed herein unless specified otherwise, the electrode comprises a hydrogel component.

In a fifty-fifth aspect of the present disclosure, which may be combined with any other aspect listed herein unless specified otherwise, the sensor comprises at least four electrodes.

In a fifty-sixth aspect of the present disclosure, which may be combined with any other aspect listed herein unless specified otherwise, the system further includes an electrical impedance circuit configured to electrically connect to each of the four electrodes.

In a fifty-seventh aspect of the present disclosure, which may be combined with any other aspect listed herein unless specified otherwise, the electrical impedance circuit is configured to inject electrical current into a first set of electrodes, and measure bio-electric signals from a second set of electrodes.

In a fifty-eighth aspect of the present disclosure, which may be combined with any other aspect listed herein unless specified otherwise, the electrical impedance circuit is configured to process the bio-electric signals from the second set of electrodes to generate a time-dependent impedance waveform.

In a fifty-ninth aspect of the present disclosure, which may be combined with any other aspect listed herein unless specified otherwise, the processing system receives the time-dependent impedance waveform, and the first algorithm operated by the processing system processes the time-dependent impedance waveform to determine a value of heart rate.

In a sixtieth aspect of the present disclosure, which may be combined with any other aspect listed herein unless specified otherwise, the processing system receives the time-dependent impedance waveform, and the first algorithm operated by the processing system processes the time-dependent impedance waveform to determine a value of respiration rate.

In a sixty-first aspect of the present disclosure, which may be combined with any other aspect listed herein unless specified otherwise, the processing system receives the time-dependent impedance waveform, and the first algorithm operated by the processing system processes the time-dependent impedance waveform to determine a value of fluids.

In a sixty-second aspect of the present disclosure, which may be combined with any other aspect listed herein unless specified otherwise, the processing system receives the time-dependent impedance waveform, and the second algorithm operated by the processing system processes the time-dependent impedance waveform to determine when the liquid solution provided by the catheter is delivered outside of the vein.

In a sixty-third aspect of the present disclosure, which may be combined with any other aspect listed herein unless specified otherwise, the sensor is a temperature sensor.

In a sixty-fourth aspect of the present disclosure, which may be combined with any other aspect listed herein unless specified otherwise, the temperature sensor is one of a thermistor, thermocouple, resistance temperature detector, thermometer, optical sensor, and thermal flow sensor.

In a sixty-fifth aspect of the present disclosure, which may be combined with any other aspect listed herein unless specified otherwise, the system further includes a temperature-measuring circuit configured to electrically connect to the temperature sensor.

In a sixty-sixth aspect of the present disclosure, which may be combined with any other aspect listed herein unless specified otherwise, the temperature-measuring circuit is configured to process the signals from the temperature sensor to generate a time-dependent temperature waveform.

In a sixty-seventh aspect of the present disclosure, which may be combined with any other aspect listed herein unless specified otherwise, the processing system receives the time-dependent temperature waveform, and the first algorithm operated by the processing system processes the time-dependent temperature waveform to determine a value of skin temperature.

In a sixty-eighth aspect of the present disclosure, which may be combined with any other aspect listed herein unless specified otherwise, the processing system receives the time-dependent temperature waveform, and the first algorithm operated by the processing system processes the time-dependent temperature waveform to determine a value of core temperature.

In a sixty-ninth aspect of the present disclosure, which may be combined with any other aspect listed herein unless specified otherwise, the processing system receives the time-dependent temperature waveform, and the second algorithm operated by the processing system processes the time-dependent temperature waveform to determine when the liquid solution provided by the catheter is delivered outside of the vein.

In a seventieth aspect of the present disclosure, which may be combined with any other aspect listed herein unless specified otherwise, the system further includes a motion sensor.

In a seventy-first aspect of the present disclosure, which may be combined with any other aspect listed herein unless specified otherwise, the motion sensor is one of an accelerometer or gyroscope.

In a seventy-second aspect of the present disclosure, which may be combined with any other aspect listed herein unless specified otherwise, the motion sensor is configured to generate a time-dependent motion waveform.

In a seventy-third aspect of the present disclosure, which may be combined with any other aspect listed herein unless specified otherwise, the processing system is further configured to receive the time-dependent motion waveform and analyze it and the signals from the sensor to determine the physiological parameter.

In a seventy-fourth aspect of the present disclosure, which may be combined with any other aspect listed herein unless specified otherwise, the processing system is further configured to receive the time-dependent motion waveform and analyze it and the signals from the sensor to determine when the liquid solution provided by the catheter is delivered outside of the vein.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been selected for readability and instructional purposes, and not to limit the scope of the inventive subject matter.

### BRIEF DESCRIPTION OF THE DRAWINGS

FIG. 1 is a drawing of the IVDS according to the invention;

FIG. 2A is a graph showing time-dependent motion, temperature, IMP, and PVP waveforms measured before and after IV infiltration using the IVDS of FIG. 1;

FIGS. 2B, 2C, and 2D are schematic drawings showing how, respectively, PVP, IMP, and temperature sensors within the IVDS sensor measure corresponding signals from a patient;

FIG. 3A is a graph of the time-dependent PVP waveform of FIG. 2A;

FIGS. 3B and 3C are graphs of the time-dependent PVP waveform of FIG. 3A measured, respectively, before and after IV infiltration;

FIG. 4A is a graph of SYS BP measured by both a cuff-based system and a cuffless technique of the prior art based on pulse transit time;

FIG. 4B is a graph of SYS BP measured by both a catheter inserted into a porcine subject's artery and a technique for processing PVP waveforms used in the IVDS of FIG. 1;

FIG. 5 is a schematic drawing of the IVDS of FIG. 1 and an infusion pump attached to a patient in a hospital bed;

FIG. 6 is a schematic drawing indicating how the IVDS of FIG. 1 attaches to a patient and measures PVP waveforms;

FIG. 7A is an image of a PVP-conditioning circuit board used in the IVDS of FIG. 1 to amplify and condition PVP signals generated by the sensor shown in FIG. 6B;

FIG. 7B is a photograph of the PVP-conditioning circuit board indicated by the image shown in FIG. 7A;

FIG. 8 is an electrical schematic describing the PVP-conditioning circuit board of FIGS. 7A and 7B featuring circuits for filtering, amplifying, and digitizing PVP-AC and PVP-DC waveforms;

FIG. 9A is a time-dependent plot of a first PVP-AC waveform measured after a first amplifier stage described by the electrical schematic of FIG. 8;

FIG. 9B is a time-dependent plot of a second PVP-AC waveform measured after a second amplifier/filter stage described by the electrical schematic of FIG. 8;

FIG. 9C is the electrical schematic of FIG. 8, further illustrating various measurement locations;

FIG. 10A is a graph of a time-dependent PVP waveform featuring ‘beatpicks’ generated by a conventional beatpicking algorithm;

FIG. 10B is a graph of a time-dependent PVP waveform featuring beatpicks generated by a beatpicking algorithm used in the IVDS of FIG. 1;

FIG. 11A is a graph of a time-dependent arterial BP waveform featuring beatpicks generated by a beatpicking algorithm indicated by FIG. 10B;

FIG. 11B is a graph of a time-dependent arterial BP waveform measured from a relatively short time segment of FIG. 11A and indicating both cardiac and respiratory components;

FIG. 11C is a graph of a time-dependent PVP waveform featuring beatpicks generated by a beatpicking algorithm indicated by FIG. 10B;

FIG. 11D is a graph of a time-dependent PVP waveform measured from a relatively short time segment of FIG. 11C indicating both cardiac and respiratory components;

FIGS. 12A-E are graphs of time-dependent arterial BP and PVP waveforms measured from five different porcine subjects;

FIG. 13A is a graph showing the relationship between pressure and volume changes for human veins and arteries;

FIG. 13B is a graph showing how the relationship between pressure and volume changes for human veins and arteries during periods of vascular smooth muscle contraction (e.g., during respiration), which reduces vascular compliance;

FIGS. 14A and 14B are graphs of beatpicks generated from, respectively, time-dependent arterial BP and PVP waveforms that are both unfiltered and filtered to remove a respiratory artifact;

FIG. 15 is a schematic drawing of the IVDS of FIG. 1 connected through Bluetooth® to both a BP cuff that calibrates its BP measurement and an infusion pump that displays information it generates;

FIG. 16 is a graph of time-dependent motion and PVP waveforms measured while a subject's arm was disposed in different positions;

FIG. 17 is a flow chart indicating an algorithm used by the IVDS of FIG. 1 to determine SYS and DIA values from PVP waveforms;

FIGS. 18A-E are graphs of time-dependent SYS BP values measured from both an arterial BP waveform and a PVP waveform processed with the algorithm indicated in FIG. 17;

FIG. 19 is a graph of derived from information plotted in the graphs in FIG. 18A-E that indicates agreement between SYS values measured from both an arterial BP waveform and a PVP waveform processed with the algorithm indicated in FIG. 17;

FIG. 20 is a graph showing time-dependent motion, temperature, IMP, and PVP waveforms measured from a patient undergoing different postures and types of motion; and,

FIGS. 21A and 21B are graphs showing, respectively, time-dependent PPG and IMP waveforms measured with the IVDS of FIG. 1 and used to calculate vital signs from a patient.

### DETAILED DESCRIPTION

### 1. Overview

Although the following text sets forth a detailed description of numerous different embodiments, it should be understood that the legal scope of the invention described herein is defined by the words of the claims set forth at the end of this patent. The detailed description is to be construed as exemplary only; it does not describe every possible embodiment, as this would be impractical, if not impossible. One of ordinary skill in the art could implement numerous alternate embodiments, which would still fall within the scope of the claims.

### 2. IVDS

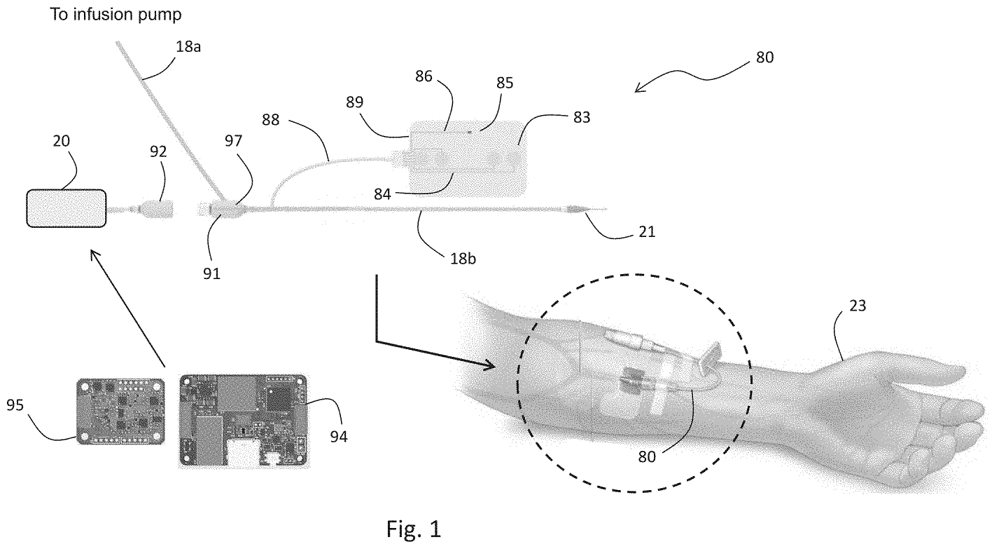

Referring to FIG. 1, an IVDS 80 according to the invention provides three primary functions: 1) it secures an IV catheter 21 to a body component (e.g., an arm 23) of a patient to deliver fluids (e.g., saline, medication dissolved in saline) into their venous system; 2) it simultaneously detects problems associated with the IV catheter (i.e., infiltration, extravasation, and occlusion) that can reduce the efficacy of such delivery; and 3) it simultaneously measures biometric signals that, once processed, yield physiological parameters from the patient (e.g., HR, RR, TEMP, SpO2), and most notably SYS and DIA. Computer systems in the hospital can analyze these physiological parameters and subsequently influence the delivery of fluids to the patient, thus enabling a ‘closed-loop’ system that can potentially improve patient care.

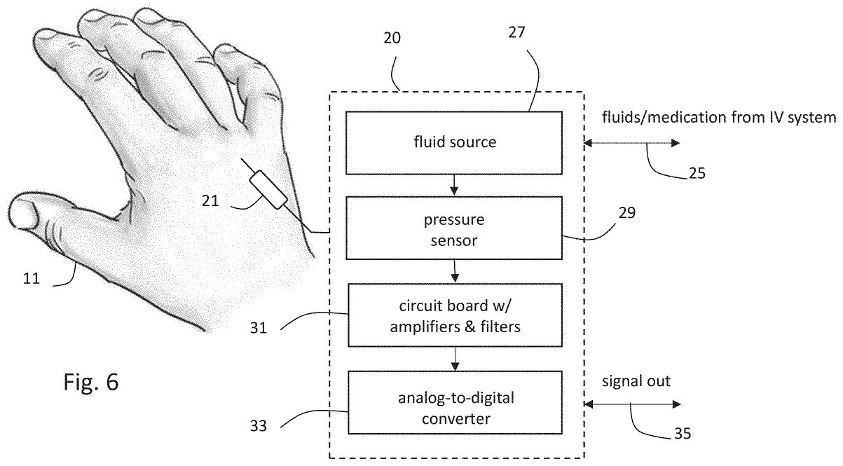

The IVDS features a flexible, breathable polymeric base 89—similar that used in a large bandage—with a biocompatible adhesive on one side that secures the IV catheter 21 in place. In FIG. 1 the IV catheter 21 is exposed, but during a medical procedure it is inserted into a vein within the patient's arm 23. The polymeric base 89 includes a set of electrodes 83 (typically four) composed of a conventional hydrogel material; these connect through a first set of embedded electrical traces 84 in a cable 88 that ultimately leads to an impedance circuit within an electronics module 94 enclosed within an arm-worn housing 20. The electrodes 83 are typically arranged in a linear configuration and disposed along the span of the vein; alternatively, they can be arranged in a ‘square’ configuration that positions them in the four corners of the polymeric base 89. The electronics module 94 features a printed circuit board that, in turn, supports various electronic components (e.g., circuits for signal amplification and power management; an accelerometer for characterizing patient motion; a microprocessor and associated memory for processing sensor-generated information; a wireless transmitter for transmitting information to an external display; and a rechargeable battery for powering the system) that enable the above-described measurements. Located proximal to the electronics module 94 is a PVP-conditioning circuit board 95, described in more detail below with references to FIGS. 6-9, that includes a series of analog amplifiers and filters that process signals from the pressure sensor 97, which is typically located in a first connector 91. The PVP-conditioning circuit board 95 generates PVP-AC and PVP-DC signals for follow-on processing.

During use, the set of electrodes 83 attach to the patient's skin to measure bio-electric signals that, once processed with the electronics module 94, indicate the electrical impedance of tissue disposed underneath the polymeric base 89. The polymeric base 89 additionally includes a temperature sensor 85 that connects through a second set of electrical traces 86 to the cable 88, which ports electrical signals from the electrodes 83 and temperature sensor 85 to the first connector 91. The first connector 91 mates with a second connector 92 that ports the electrical signals to the electronics module 94 within the arm-worn housing 20. Typically, the second connector 92, electronics module 94, and arm-worn housing are considered ‘reusable’ components of the IVDS, whereas the other components shown in FIG. 1 are considered ‘disposable’ components.

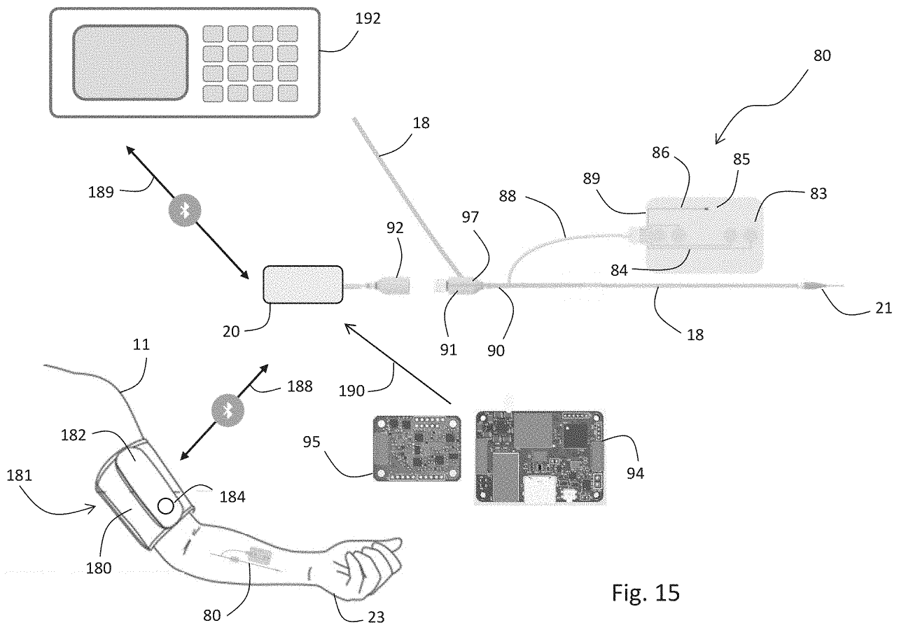

During use, the catheter 21 inserts into the patient's vein and connects to an infusion pump (not shown in the figure but indicated in FIG. 15) through a segment of IV tubing 18a. A portion of the tubing 18b passes through the connector 91, which features the small pressure sensor 97 that measures pressure of a ‘fluid column’ within the segment of the tubing 18b. Small pressure fluctuations within the patient's venous system, in turn, modulates pressure within the fluid column. The pressure sensor 97 measures these pressure fluctuations, and in response generates electrical signals that pass through the first connector 91, second connector 92, and into the electronics module 94, where they are conditioned (e.g., filtered, amplified) with the PVP-conditioning circuit board 95 and then processed, as described in more detail below, to simultaneously measure parameters related to the performance of the IV system and the patient's physiology.

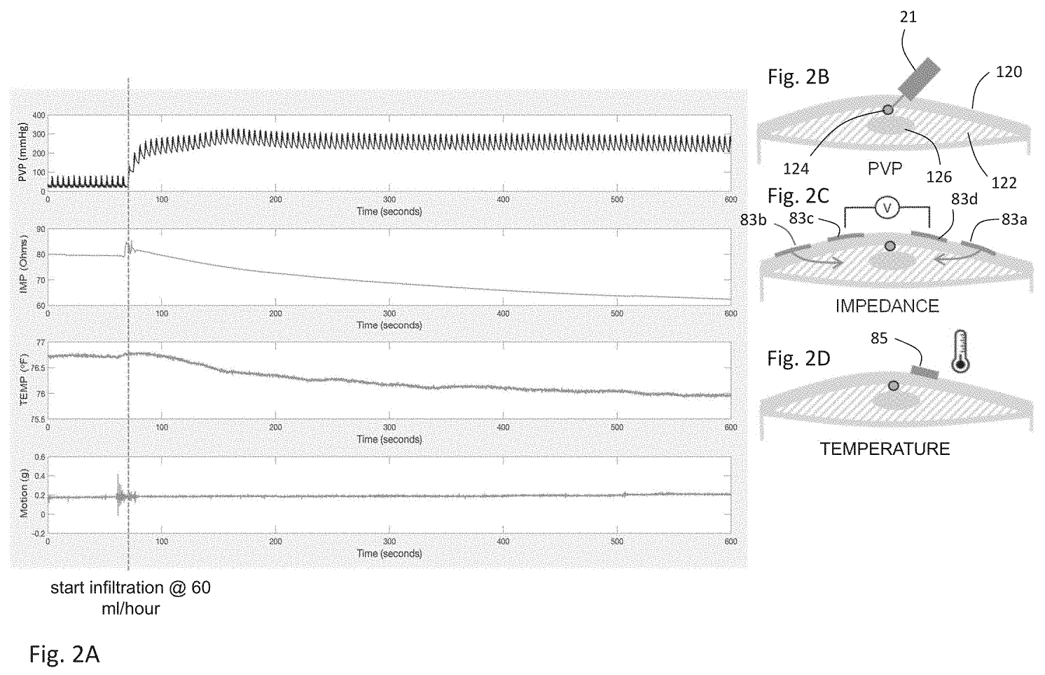

FIGS. 2A-D indicate how the IVDS shown in FIG. 1 can characterize infiltration from the catheter 21. More specifically, FIG. 2A shows a graph of time-dependent motion, temperature, IMP and PVP waveforms measured by the IVDS. For these measurements, an infusion pump delivering fluids at a rate of 60 ml/hour was connected to a patient outfitted with a special arm-worn rig that facilitated infiltration. Sensors measuring temperature, IMP, PVP, and patient motion were attached directly to the arm-worn rig and connected through cables similar to those described for FIG. 1 to an electronics module within an arm-worn housing 20.

As indicated in the graph, infiltration was initiated at approximately 60 seconds. Fluctuations in the motion waveform indicate that, at this time, the patient moved, thereby causing the catheter 21 to push from within a vein 124 in the arm-worn rig into the surrounding tissue 122, which is typically composed of agar, a conductive, gelatinous material. The arm-worn rig additionally includes synthetic components representing a bone 126 and skin 120. Additionally, a control circuit and motorized pump (not shown in the figure) attaches to the vein and pumps a conductive, blood-like liquid at a ‘heart rate’ that is approximately 60 beats/min.

Referring to FIG. 2C, electrodes 83a-d connect to the skin 120 of the arm-worn rig, and sense signals that are processed with the impedance circuit within the electronics module to determine the electrical impedance of tissue underneath them. More specifically, for the impedance measurement outer electrodes 83a, 83b inject a high-frequency (typically between 20-100 kHz), low-amperage (typically between 10-1000 □A) current through the skin 120 and into the surrounding tissue 122. The injected current propagates into the surrounding tissue, which has an electrical conductivity matched to human tissue. The resistance of the surrounding tissue impacts current flow, which is manifested by a voltage drop that is measured by a pair of inner electrodes 83c, 83d. This voltage drop is digitized by the impedance system to yield the IMP waveform.

As shown in the graph in FIG. 2A, prior to infiltration the IMP waveform is relatively stable. Immediately following infiltration, it steadily decreases in value; this trend continues for at least 600 seconds, at which point the test was terminated. This is because prior to infiltration, the infusion pump delivers fluid (which in this case is conductive) directly into the vein, where flow of the blood-like liquid driven by the control circuit and motorized pump rapidly whisks it away, thereby minimizing its impact on the impedance of the surrounding tissue 122. However, after the catheter is pushed through the vein 124, fluid from the infusion pump flows directly into the surrounding tissue 122. And because the fluid is conductive, it lowers the impedance (i.e., resistance) of the tissue, thereby causing the IMP waveform to gradually decrease.

A similar situation exists for the temperature waveform, as shown in the graph in FIG. 2A. Here, the temperature of the fluid delivered from the infusion pump is kept approximately 20° F. colder than the components within the arm-worn rig; this is meant to mimic the situation occurring in typical hospital environments, wherein fluids and medications are typically kept at room temperature (approximately 72° F.) when delivered with IV systems, whereas the human body is more than 20° F. warmer. Relatively lower temperature fluid from the infusion pump infiltrating from the vein 124 into the surrounding tissue 122 causes the temperature of the surrounding tissue to drop. It is measured by the temperature sensor 85, as indicated in FIG. 2D. As indicated in FIG. 2A, this results in a temperature waveform that slowly decreases after infiltration in a manner similar to the IMP waveform.

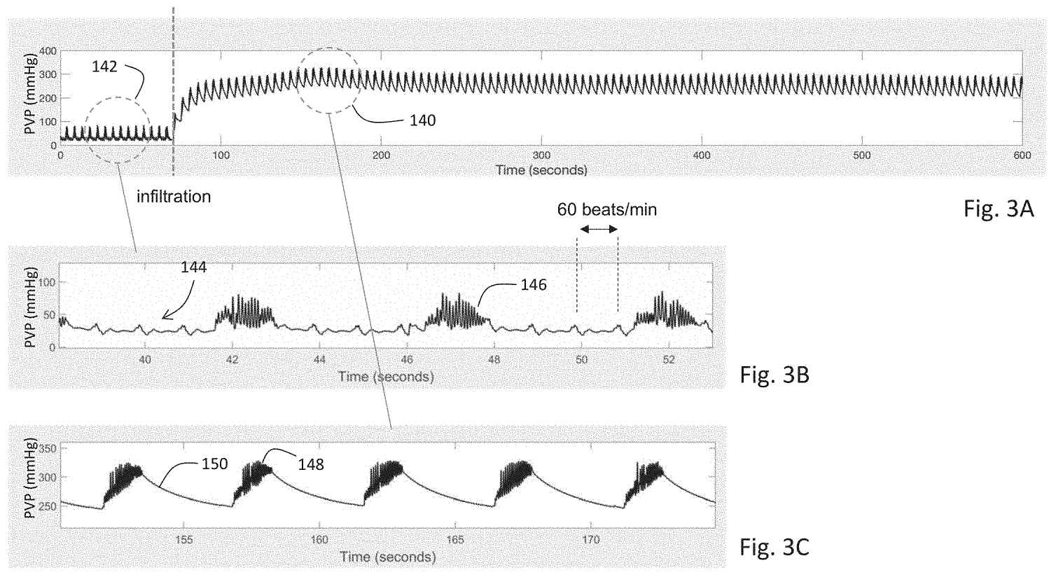

The PVP waveform is measured with a pressure sensor configured as shown in FIG. 1 and features several signal components that change following infiltration. As indicated by FIGS. 2A, 2B, and 3A-3C, the PVP waveform, like the temperature and IMP waveforms, is relatively stable prior to infiltration. As shown in FIG. 3B, which is a close-up view of the PVP waveform taken from a time-period within the circle 142 in FIG. 3A, prior to infiltration the PVP waveform features a set of small, periodic pulses 144, which represent flow of the blood-like liquid driven by the control circuit and motorized pump through the vein. Note that in FIG. 3B, the periodic pulses 144 occur at a frequency of approximately 60 beats/min, as set by the control circuit. Additionally, prior to infiltration, the PVP waveform features periods of high-frequency noise 146 which are caused by the infusion pump, which periodically delivers liquid to the vein at a rate of 60 mL/hour.

Several things happen to the PVP waveform after infiltration. Referring specifically to FIGS. 3A and 3C, the latter of is a close-up view of the PVP waveform taken from a time-period within the circle 140 in FIG. 3A, immediately following infiltration fluid from the infusion pump is no longer delivered to the vein and flows into the surround tissue. This manifests as a rapid pressure increase from around 20 mmHg prior to infiltration to nearly 300 mmHg after infiltration. Additionally, because the catheter is no longer disposed in the vein, the heartbeat-induced pulses evident in FIG. 3B are no longer present. Moreover, because the surrounding tissue is decidedly less efficient at whisking away fluid, each bolus delivered by the infusion pump causes a pressure pulse 150 that rises from a baseline of about 250 mmHg to a peak of about 300 mmHg, before decaying away in a manner that represents the fluid diffusing into the surrounding tissue. Each pressure pulse 150 is caused entirely by the infusion pump, and thus features high-frequency noise 148, similar to component 146 in FIG. 3B.

In summary, within the PVP waveform there are several signal components—rapid rise in pressure, heartbeat-induced pulses and their subsequent disappearance, large pressure pulses—that an algorithm can process to characterize IV infiltration. Such an algorithm can collectively process PVP waveforms along with IMP, temperature, and motion waveforms to better detect this event. Additionally, other sensors, such as those that measure optical, acoustic, bio-reactance, and other waveforms, can be added to the IVDS to better detect this event.

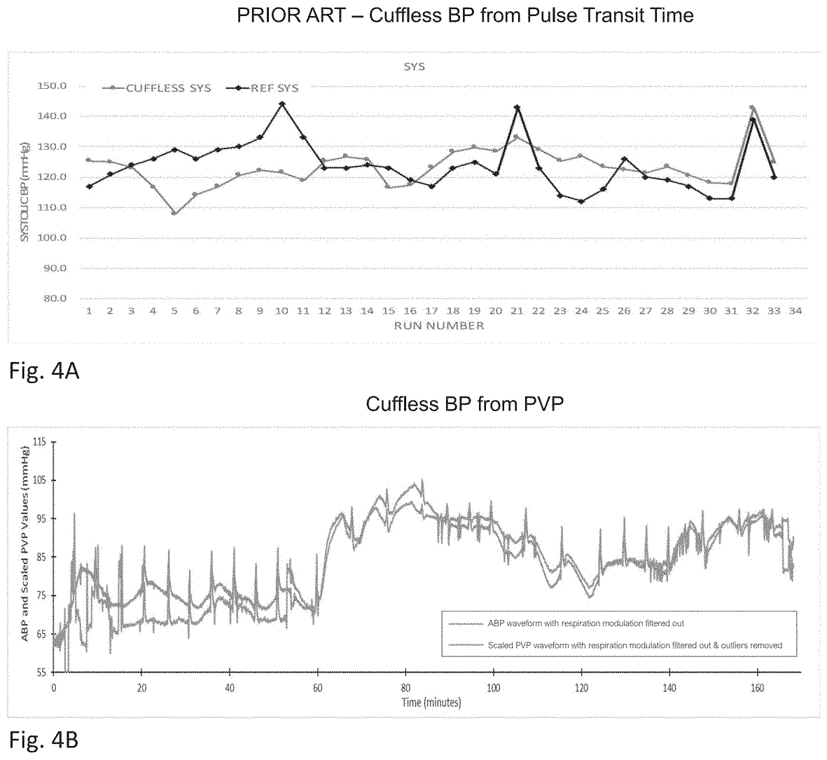

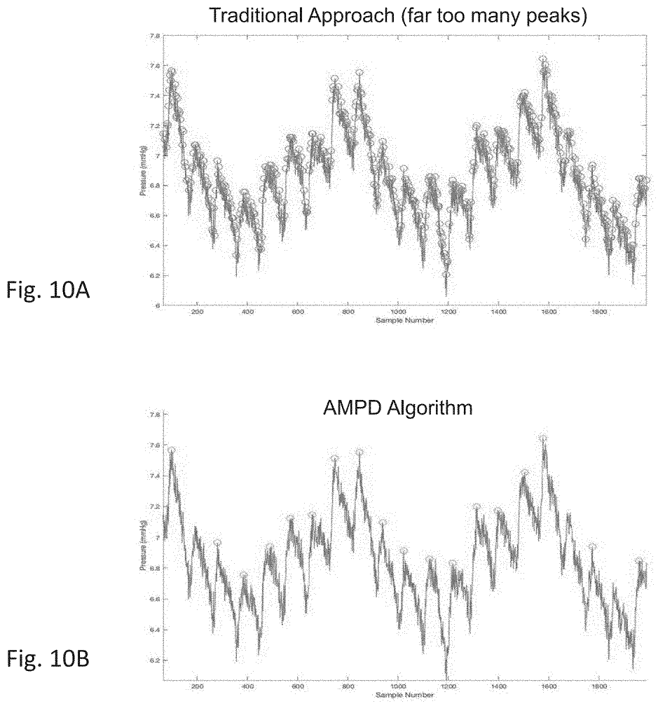

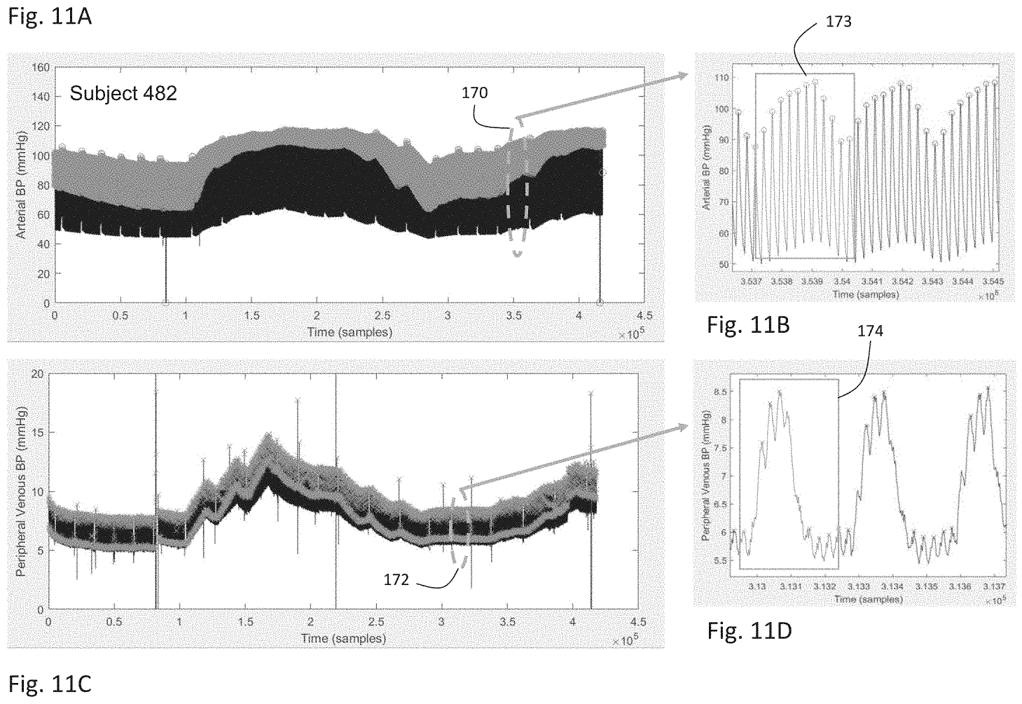

Additional algorithms can also process the PVP waveform, which represents a venous pressure, to determine arterial blood pressure, as indicated by FIGS. 10-13, and 16-18, and the associated descriptions of these figures below. FIG. 4B indicates the accuracy of such a measurement of blood pressure, particularly when compared to ‘cuffless’ approaches of the prior art based on technologies such as PTT and PAT. For example, the graph shown in FIG. 4A shows typical results for SYS as measured using a PTT-based approach. The figure indicates reasonable correlation between a reference measurement (in this case made with a pair of clinicians measuring blood pressure using auscultation). However, the PTT-based approach is relatively insensitive to rapid swings in blood pressure that the reference measurement detects. In contrast, FIG. 4B shows continuous arterial blood pressure (specifically SYS) measured from a subject using an in-dwelling arterial line, along with blood pressure calculated from a corresponding PVP waveform measured simultaneously from the same subject using an algorithm described herein. Here, the PVP-determined value of SYS is highly correlated to that of the reference measurement, even for rapid, short-term rises and drops in blood pressure. Similar measurements are described in more detail below, particularly with reference to FIGS. 11, 12, 14 and 18. This indicates that the IVDS described herein, in addition to securing a catheter in place, can additionally measure BP values while simultaneously detecting IV infiltration.

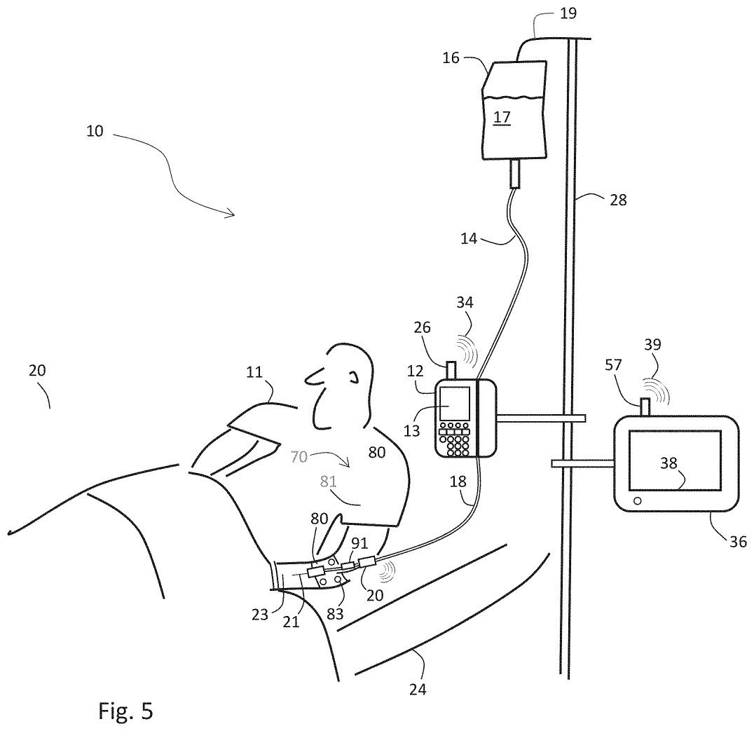

FIG. 5 shows how the IVDS 80 system described herein can be incorporated into a hospital setting to measure a patient 11. Here, the IVDS 80 is deployed within a system 10 featuring an IV system 19 to characterize IV-related parameters and vital signs from a patient 11 deposed in a hospital bed 24. The arm-worn housing 20 within the IVDS 80 encloses the electronics module and PVP-conditioning circuit board that is configured to amplify, filter, and digitize PVP signals. The arm-worn housing 20 terminates with a venous catheter 21 inserted into a vein in the patient's hand or arm. A remote processor 36 (e.g., a tablet computer or device with comparable functionality) connects to the arm-worn housing 20 through a through a wireless interface (e.g., Bluetooth®). In embodiments, the remote processor 36 can also connect to the arm-worn housing through wired (e.g., cable) means; this may be used, for example, to charge the Li-ion battery within the electronics module. During a measurement, the remote processor 36 receives information from the IV system 19 and the IVDS 80, and collectively analyzes this as described in detail herein to monitor the patient.

The IV system 19 features a bag 16 containing pharmaceutical compounds and/or fluid (herein “medication” 17) for the patient. The bag 16 connects to an infusion pump 12 through a first tube 14. A standard IV pole 28 supports the bag 16, the infusion pump 12, and the remote processor 36. A display 13 on the front panel of the infusion pump 12 indicates the type of medication delivered to the patient, its flow rate, measurement time, etc. Medication 17 passes from the bag 16 through the first tube 14 and into the infusion pump 12. From there, it is metered out appropriately, and passes through a second tube 18, through the connector 91 featuring a pressure sensor, and finally through the venous catheter 21 and into the patient's venous system 23. The arm-worn housing 20 connects to the connector 91 and is typically affixed to the patient's arm or hand, e.g., using an adhesive such as medical tape or a disposable electrode.

The venous catheter 21 may be a standard venous access device, and thus may include a needle, catheter, cannula, or other means of establishing a fluid connection between the catheter 21 and the patient's peripheral venous system 23. The venous access device may be a separate component connected to the venous catheter 21, or may be formed as an integral portion of it. In this way, the IV system 19 supplies the medication 17 to the patient's venous system 23 while the IVDS 80, which features a pressure-measuring system and described in more detailed below, simultaneously measures signals related to the patient's PVP and vital signs.

Importantly, and as described in more detail below, the IVDS 80 is designed so that it is in constant ‘fluid connection’ with the patient's circulatory system (and particularly the venous system) while being deployed close to (or directly on) the patient's body. It features electronic systems for measuring analog pressure signals within the patient's venous system to generate PVP waveforms, and then amplifying and filtering these to optimize their signal-to-noise ratios. An analog-to-digital converter within the arm-worn housing digitizes the analog PVP waveforms prior to transmitting them through the cable, thereby minimizing any noise (caused, e.g., by the cable's motion) that would normally affect transmitted analog signals and ultimately introduce inaccuracies into values (e.g., values of BP, HR, RR, F0 and F1) measured downstream. Notably, this design provides a relatively short conduction path between where the PVP waveforms are first detected and then processed and digitized; ultimately this results in signals that are more likely to yield highly accurate values of wedge pressure (and in embodiments pulmonary arterial pressure, and particularly the diastolic component on this pressure, blood volume and other fluid-related parameters).

FIG. 6 shows in more detail the arm-worn housing 20, its method of operation, and how its internal components (the electronics module and PVP-conditioning circuit board) function therein. The housing 20 is designed to rest comfortably close to or on the patient while: 1) allowing fluids (and/or medication) from the IV system to flow (as indicated by arrow 25) into the patient's venous system (box 27); 2) measuring pressure signals from the patient's venous system with a pressure sensor (box 29); 3) filtering/amplifying the pressure signals with circuits functioning as analog amplifiers and filters (box 31); 4) digitizing the filtered/amplified signals with an analog-to-digital converter (box 33); and 5) transmitting the digitized signals using Bluetooth® transceiver for further processing by the remote processor (arrow 35).

### 3. PVP-Conditioning Circuit Board

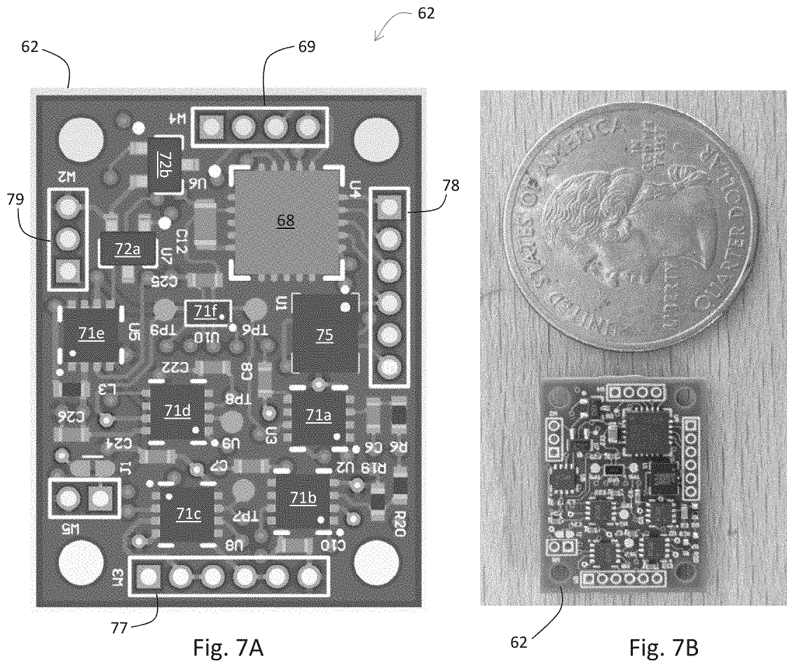

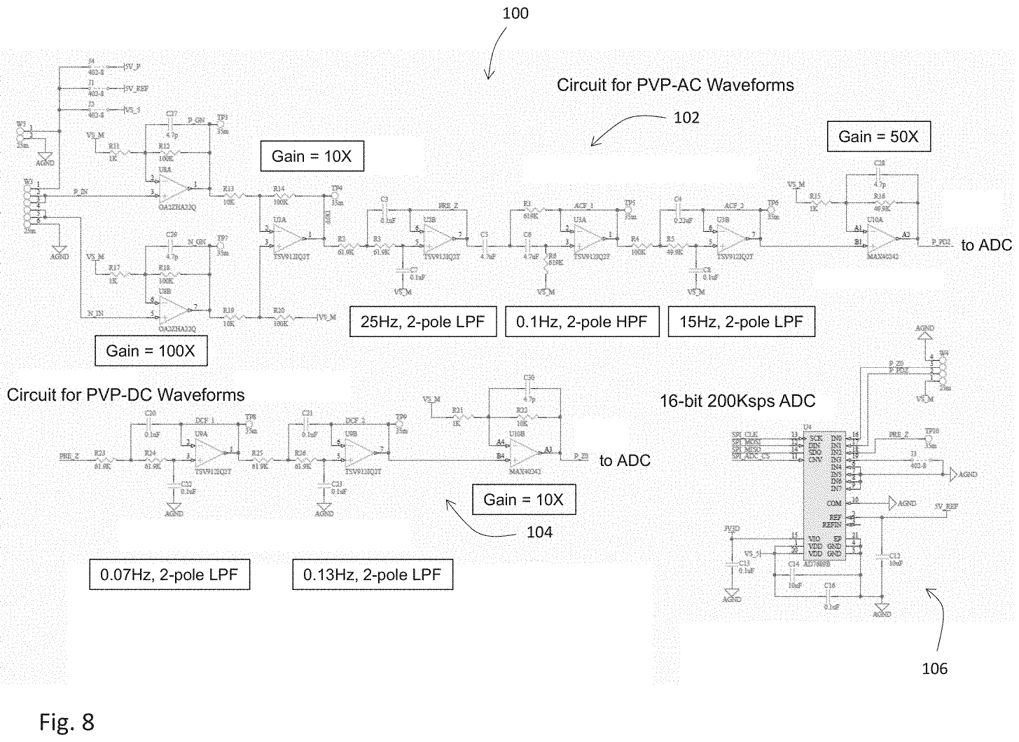

FIGS. 7A and 7B show, respectively, an image and photograph of the PVP-conditioning circuit board 62 within the arm-worn housing. The circuit board 62 was fabricated according to an electrical schematic, shown in FIG. 8 (specifically component 100) and described in more detail below. The circuit board 62 shown in the figure is a 4-layer fiberglass/metal structure that includes metal pads soldered to, among other components, an analog-to-digital converter 68, accelerometer 75, operational amplifiers 71a-f, and power regulators 72a-b. More specifically, operational amplifiers 71a-d make up analog high and low-pass filters, and operational amplifiers 71e-f and power regulators 72a-b collectively regulate power levels for the various components in the circuit board 62. The accelerometer 75 measures motion of the circuit board 62 and, in doing this, any part of the patient's body it is attached to. The analog-to-digital converter 68 digitizes analog PVP waveforms after they have been filtered and converts them into digital waveforms with 16-bit resolution and a maximum digitization rate of 200 Ksamples/second (herein “Ksps”).

The PVP-conditioning circuit board 62 additionally includes sets of metal-plated holes that support a 4-pin connector 69, two 6-pin connectors 77, 78, and a 3-pin connector 79. More specifically, connector 69 connects directly to the pressure transducer, where it receives a common ground signal and analog PVP waveforms representing pressure in the patient's venous system. These waveforms are filtered and digitized as described in more detail, below. Through the connector 79 the circuit board receives power (+5V, +3.3V, and ground) from an external power supply, e.g., a battery or power supply located in the arm-worn housing. These power levels may be different in other embodiments of the invention. Digital signals and a corresponding ground from the analog-to-digital converter 68 are terminated at connector 78; they leave the circuit board 62 at this point, e.g., through cable segment 37 shown in FIG. 2C. Connector 77 is used primarily for testing and debugging purposes, and allows analog PVP signals, once they pass through analog high and low-pass filters, to be measured with an external device such as an oscilloscope.

The PVP-conditioning circuit board 62 typically connects to the electronics module through a serial interface (e.g., SPI, I2C), which includes components for processing, storing, and transmitting data that are digitized by the analog-to-digital converter 68. For example, electronics module typically includes a microprocessor, microcontroller, or similar integrated circuit, and can additionally provide analog and digital circuitry for the IVDS. In embodiments, the microprocessor or microcontroller thereon can operate computer code to process PVP-AC, PVP-DC, PPG, IMP, BP, and other time-dependent waveforms to determine vital signs (e.g., HR, HRV, RR, BP, SpO2, TEMP), hemodynamic parameters (CO, SV, FLUIDS), components of PVP waveforms (e.g., F0, F1, and amplitudes and energies associated thereto), and associated parameters (e.g., wedge pressure, central venous pressure, blood volume, fluid volume, and pulmonary arterial pressure) related to the patient's fluid status. “Processing” by the microprocessor in this way, as used herein, means using computer code or a comparable approach to digitally filter (e.g., with a high-pass, low-pass, and/or band-pass filter), transform (e.g., using FFT, CWTs, and/or DWTs), mathematically manipulate, and generally process and analyze the waveforms and parameters and constructs derived therefrom with algorithms known in the art. Examples of such algorithms include those described in the following co-pending and issued patents, the contents of which are incorporated herein by reference: “NECK-WORN PHYSIOLOGICAL MONITOR”, U.S. Ser. No. 14/975,646, filed Dec. 18, 2015; “NECKLACE-SHAPED PHYSIOLOGICAL MONITOR”, U.S. Ser. No. 14/184,616, filed Aug. 21, 2014; and “BODY-WORN SENSOR FOR CHARACTERIZING PATIENTS WITH HEART FAILURE”, U.S. Ser. No. 14/145,253, filed Jul. 3, 2014.

In related embodiments, the electronics module can include both flash memory and random-access memory for storing time-dependent waveforms and numerical values, either before or after processing by the microprocessor. In still other embodiments, the circuit board can include Bluetooth® and/or Wi-Fi transceivers for both transmitting and receiving information.

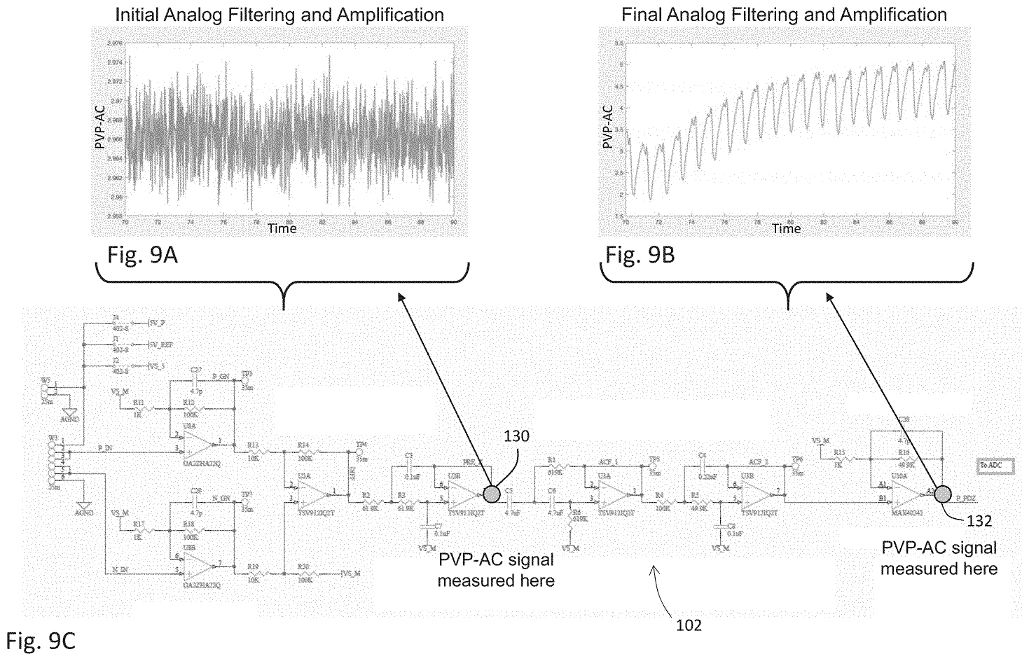

PVP waveforms measured with the system described herein feature signal components that relate to heartbeat and respiratory events that may vary rapidly with time. FIG. 9 shows examples of PVP-AC waveforms, and how they are amplified and conditioned by the PVP-conditioning circuit board 62 in the arm-worn housing 20 to improve their signal-to-noise ratio.

More specifically, PVP waveforms typically have signal levels in the 5-50 □V range, a relatively weak amplitude that can be difficult to process. Such signals have been described previously, e.g., in U.S. patent application Ser. No. 16/023,945 (filed Jun. 29, 2018 and published as U.S. Patent Publication 2019/0000326); U.S. patent application Ser. No. 14/853,504 (filed Sep. 14, 2015 and published as U.S. Patent Publication No. 2016/0073959), and PCT Application No. PCT/US16/16420 (filed Feb. 3, 2016 and published as WO 2016/126856). The contents of these pending patent applications are incorporated herein by reference. During a measurement, as described in these documents, a pressure sensor proximal to the patient measures the PVP waveform and generates corresponding analog signals; these typically pass through a relatively long cable, and are amplified, filtered, and digitized with a system located remotely from the patient. However, because PVP waveforms are so weak and characterized by low signal-to-noise ratios, they can be extremely difficult to measure. It is therefore advantageous to digitize these signals before they propagate through a long, ‘lossy’ cable.

FIG. 8 shows a schematic 100 of the circuit board 62 shown in FIGS. 7A-B. The schematic 100 includes: 1) a first set of circuit elements 102 designed to amplify and filter PVP-AC waveforms; 2) a second set of circuit elements 104 designed to amplify and filter PVP-DC waveforms; and 3) a 16-bit, 200 Ksps analog-to-digital converter 106 to digitize both the PVP-AC and PVP-DC waveforms.

More specifically, the circuit described by the schematic 100 is designed to serially perform the following function on incoming PVP waveforms:

Incoming PVP Waveforms

1) Amplify the signal with 100× gain using a zero-drift amplifier

2) Differentially amplify the signal with an additional 10× gain

3) Filter the amplified signals with a 25 Hz, 2-pole low-pass filter

This first portion of the circuit provides roughly 1000× combined gain for the incoming PVP waveforms, thereby amplifying the input signal (which is typically in the □V range) to a larger signal (in the mV range). The follow-on low-pass filter removes any high-frequency noise. Ultimately these steps facilitate processing of both the PVP-AC and PVP-DC waveforms, as described below.

In the descriptions provided herein, the term ‘differentially amplify’ refers to a process wherein the circuit measures the difference between positive (P_IN in FIG. 8) and negative (N_IN in FIG. 8) terminals. Notably, the output of the differential amplifier is a single-ended signal, zeroed at the midpoint voltage of the system. Alternatively, it could be zeroed at 0 V, although a centering point between the voltage rails generally provides a more accurate and cleaner output signal.

Likewise, the term ‘zero-drift amplifier’ refers to an amplifier that: 1) internally corrects for temperature and other forms of low-frequency signal error; 2) has very high input impedance; and 3) has very low offset voltages. The incoming signal received by a zero-drift amplifier is typically extremely small, meaning it can be subject to interference, gain shifts, or the amplifier inputs bleeding out generated current; the zero-drift architecture of the amplifier helps reduce or eliminate this.

After processing the input PVP waveforms, the circuit described by the schematic 100 is designed to serially perform the following function on PVP-AC and PVP-DC waveforms:

PVP-AC Waveforms Only
1) Filter the signal with a 0.1 Hz, 2-pole high-pass filter
2) Filter the signal with a 15 Hz, 2-pole low-pass filter
3) Amplify the signal with 50× gain

PVP-DC Signal Only
1) Filter the signal with a 0.07 Hz, 2-pole low-pass filter
2) Filter the signal with a 0.13 Hz, 2-pole low-pass filter
3) Amplify the signal with 10× gain

Both PVP-AC and PVP-DC Waveforms
1) Digitize the signals with a 16-bit, 200 Ksps Delta-Sigma analog-to-digital converter

With this level of digital signal processing, the circuit board 62 can process PVP waveforms directly on the patient's body, and more specifically signals associated with IV infiltration, respiration rate and heart rate. It performs these functions without having to send signals through an external cable, which is an approach that can add noise and other signal artifacts and thus negatively impact measurement of these parameters.

As appreciated by those skilled in the art, the circuit elements 102, 104, and 106 shown in FIG. 8 may have a comparable design that accomplishes the above-described steps with a schematic that differs slightly from that described herein. Additionally, it may include other integrated circuits and components to improve the measurement of PVP signals and thus provide added functionality. For example, the circuit board 62 may also include a temperature/humidity sensor, multi-axis accelerometer, integrated gyroscope, or other motion-detecting sensors configured to sense a motion signal associated with the patient (e.g., movement of the patient's arm, wrist, or hand). In embodiments, for example, the motion signal can be processed in tandem with the PVP waveform and used as an adaptive filter to remove motion components.

Alternatively, a motion signal measured by one of these components can be processed and compared to a pre-existing threshold value: if the signal exceeds the pre-determined threshold value, it can indicate that the patient is moving too much to make an accurate measurement; if the signal is less than the pre-determined threshold value, it can indicate that the patient is stable and that an accurate measurement can be made.

Such circuit elements 102, 104, and 106 are typically fabricated on a small, fiberglass circuit board, such as that shown in FIG. 7, characterized by dimensions designed to fit inside a small connector (e.g., component 91 in FIG. 1).

FIGS. 9A-C indicate how the circuit board 62 and associated circuit elements 102, as shown, respectively, in FIGS. 7A, 7B and 8, amplify and generally improve analog versions of the PVP-AC waveform. More specifically, FIG. 9A shows a time-dependent plot of the PVP-AC waveform measured at a location 130 within the circuit elements 102 corresponding to an initial analog filtering and amplification stage. As is clear from the figure, the signal-to-noise ratio of the PVP-AC waveform at this point is relatively weak, making it is difficult (if not impossible) to detect any features that correspond to actual physiological components, e.g., a heartbeat or respiration-induced pulse. In contrast, after passing through three additional amplification/filtering stages—1) differential amplifier with an additional 10× gain; 2) filter with a 25 Hz 2-pole low-pass filter and then a 0.1 Hz 2-pole high-pass filter and then a 15 Hz 2-pole low-pass filter; 3) amplifier with 50× gain—the signal is greatly improved. FIG. 9B shows the time-dependent waveform measured further down the circuit's amplifier chain at a second location 132: it features a relatively high signal-to-noise ratio and clear heartbeat-induced pulses (i.e., it shows a well-defined time-domain signal corresponding to HR). Such a waveform, when processed in the frequency domain as described above, would yield clear features that improve the ability of the IVDS to detect events related to IV infiltration.

Importantly and as described above, the analog signal processing indicated in FIGS. 9A-C and digitization of the PVP waveform are ideally performed as close to the signal source as possible, i.e., in the arm-worn housing. Such a configuration minimizes noise and attenuation caused by the signal propagating through a long, lossy cable (which is additionally susceptible to motion) to a remote filter/amplification circuit. Ultimately this approach yields a time-dependent waveform with the highest possible signal-to-noise ratio, thereby maximizing the accuracy to which IV infiltration and vital signs can ultimately be determined.

### 4. Blood Pressure Measurement

Even after being processing with the PVP-conditioning circuit board, PVP waveforms measured can feature low-signal to noise ratios, thereby making it difficult to extract individual heartbeat-induced pulses that are required to estimate arterial BP using the algorithm described herein. Referring to FIGS. 10A and 10B, in typical applications, heartbeat-induced pulses in time-dependent waveforms (e.g., PPG and IMP waveforms) are typically identified using algorithms that identify periodic peaks. However, such peaks can be difficult to find when the signal-to-noise ratio of the waveform is low, as indicated in FIG. 10A. In this case, the algorithm identifies multiple peaks (indicated by open circles) for each heartbeat-induced pulse. Most of these are erroneous, as only a single peak should be identified for each heartbeat-induced pulse.

FIG. 10B shows the results of an alternative beatpicking algorithm which is outlined in the following reference, the contents of which are incorporated herein by reference: Scholkmann F, Boss J, Wolf M.; “An Efficient Algorithm for Automatic Peak Detection in Noisy Periodic and Quasi-Periodic Signals”, Algorithms. 2012; 5(4):588-603. In this approach, each point in the time-dependent, pulse-containing waveform is compared to its neighbors. The algorithm iteratively increases a size of a time-dependent ‘window’ while testing for a peak. It keeps track of locations that pass the test for each window, and the width of the window sizes can be optimized based on the period of the signal (e.g., the pulse rate). The algorithm confirms ‘true’ peaks if they exist across all window sizes. FIG. 10B shows the results of this beatpicking algorithm—referred to herein as the “IVDS beatpicking algorithm”—when applied to the same PVP waveform shown in FIG. 10A. In contrast to the conventional algorithm used to process the waveform in FIG. 10A, the IVDS beatpicking algorithm correctly and singularly identifies each heartbeat-induced pulse, as shown by the open circles in FIG. 10B.

Ideally, because of the typical low signal-to-noise ratio of PVP waveforms, the IVDS described herein uses the IVDS beatpicking algorithm as described in the above-mentioned reference and demonstrated with the data shown in FIG. 10B. Typically, this algorithm is deployed using computer code such as C or C++ on a microprocessor within the IVDS's electronic module.

FIGS. 11A-D show time-dependent arterial BP and PVP waveforms measured and processed with the IVDS, and in doing so demonstrate the following key points:
Point 1: IVDS beatpicking algorithm can effectively process when both time-dependent arterial BP and PVP waveforms to identify beatpicks
Point 2: there is strong agreement between changes in time-dependent arterial and PVP waveforms, as measured and processed with the system described herein
Point 3: a patient's respiratory events modulate PVP waveforms in a significantly more pronounced manner compared to arterial BP waveforms

With regard to Point 1, the graphs in FIGS. 11A and 11C show, respectively, time-dependent arterial BP and PVP waveforms processed with the IVDS beatpicking algorithm. The open circles near to top portions of each waveform show heartbeat-induced pulses that the algorithm identifies. FIGS. 11B and 11D, which show portions of the waveforms indicated, respectively, by dashed circles 170 and 172, show both the waveforms and the beatpicks in more detail. As is clear from these data, the IVDS beatpicking algorithm successfully identifies heartbeat-induced pulses in both the arterial BP and PVP waveforms; this is particularly challenging for the PVP waveforms shown in FIGS. 11C and 11D, as signals originating from the subject's venous system have considerably less defined heartbeat-induced pulses compared to those originating from the subject's arterial system.

With regard to Points 2 and 3, comparison of the graphs shown in FIGS. 11A and 11B to those in 11C and 11D indicates there is a high degree of agreement between the time-dependent arterial BP and PVP waveforms, but the PVP waveforms are significantly more impacted by the subject's respiration. This is clearly shown in the dashed boxes 173 and 174 shown, respectively, in FIGS. 11B and 11D. In FIG. 11B—which shows the arterial BP waveform—the overall pressure is only slightly modulated by respiration. Thus, the ratio of the heartbeat-induced pulses (indicated by ‘o’ markings) to the respiration modulation is large. In contrast, in FIG. 11D—which shows the PVP waveform—the overall pressure is heavily modulated by respiration and the heartbeat-induced pulses are relatively weak. This means the ratio of heartbeat-induced pulses (indicated by ‘x’ markings) to the respiration modulation is small. Even with the respiration modulation, there is strong agreement between the two waveforms, indicating that an algorithm that digitally removes artifacts due to respiration may improve the agreement and thus commensurately improve the accuracy of BP calculated from the PVP waveform.

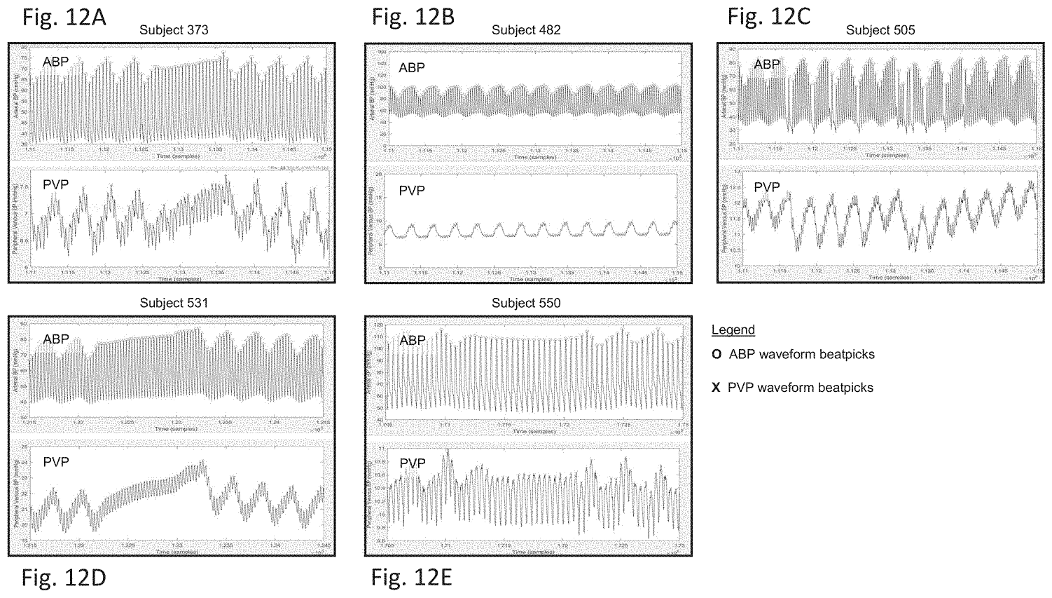

FIGS. 12A-E further demonstrate these points. Each figure shows two graphs corresponding to different porcine subjects participating in a clinical study: 1) time-dependent arterial BP waveform measured over a relatively short time segment, along with corresponding beatpicks made with the IVDS beatpicking algorithm shown with ‘o’ markers (top graph); and 2) time-dependent PVP waveform measured over with same time segment with corresponding beatpicks made with the IVDS beatpicking algorithm shown with ‘x’ markers (bottom graph). Note, for these graphs, the x-axis (“Time”) is in samples, with the sampling rate being 50 samples/second).

Data in these figures corroborate the three ‘Points’ made above: in all cases, the IVDS beatpicking algorithm is effective in locating cardiac pulses, particularly in the relatively challenging PVP waveforms. There is strong correlation between changes in the arterial BP and PVP waveforms. Moreover, in all cases, the two waveforms are both modulated by the subject's respiration in a consistent manner, with the modulation being significantly more pronounced and resulting in relatively large changes in the PVP waveforms. Importantly, the agreement between the two waveforms persists even during periods where respiratory-induced modulation is not present. For example, in FIGS. 12A and 12D, the subjects exhibit somewhat extended time periods where there is no respiration present (in both figures, roughly 1.125-1.135×10⁵ samples, or 20 seconds), but yet there is still agreement between pressure variations in the two signals.

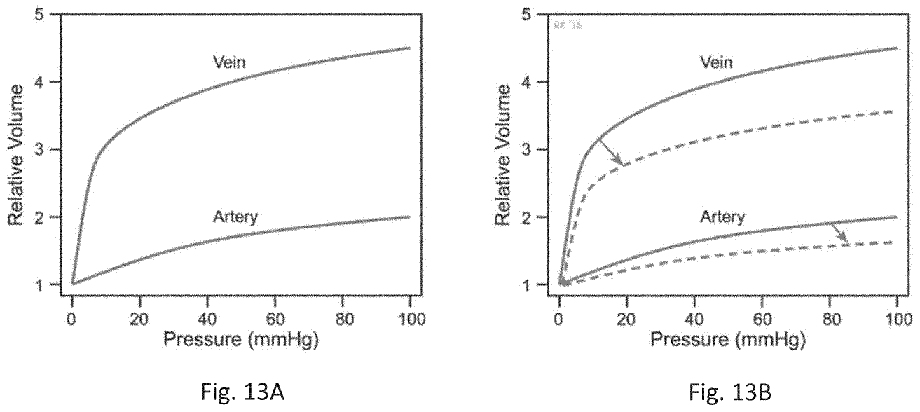

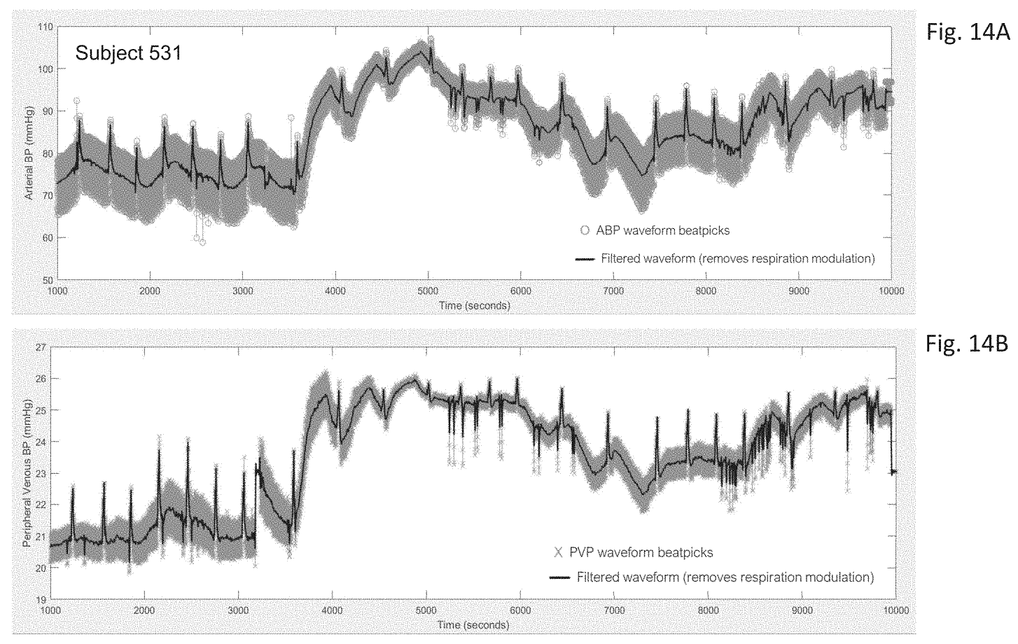

Without being bound to any particular theory, the relatively large modulation present in PVP waveforms as compared to arterial BP waveforms, as indicated by FIGS. 11 and 12, may be due to the proven theory that the compliance of a vein is about 10-20 times greater than that of an artery (see, e.g., “Cardiovascular Physiology Concepts”, by Richard E. Klabunde Ph.D., https://www.cvphysiology.com/). Referring to FIG. 13A, compliance is the ability of a blood vessel wall to expand and contract passively with changes in pressure. Typically, veins can accommodate large changes in blood volume with only a small change in pressure, meaning they have larger compliance. The greater compliance of veins is largely the result of vein collapse that occurs at pressures less than 10 mmHg. At higher pressures and volumes, venous compliance (the slope of compliance curve) is similar to arterial compliance.

There is no single compliance curve for a blood vessel. For example, as shown in FIG. 13B, vascular smooth muscle contraction, which increases vascular tone, reduces vascular compliance (dashed lines in figure) and shifts the volume-pressure relationship downward. Conversely, smooth muscle relaxation increases compliance and shifts the compliance curve upward. This is particularly important in the venous vasculature for the regulation of venous pressure and cardiac preload. Contraction of smooth muscle in arteries reduces their compliance, thereby decreasing arterial blood volume and increasing BP within the arterial system.

Compliance as described above represents the static compliance generated by expanding a vessel by a known volume and measuring the change in pressure at steady-state. Typically, the compliance of a vessel (either artery or vein) is also dependent upon the rate by which the change in volume occurs, i.e., there is a dynamic component to compliance. This is indicated in FIGS. 11 and 12 by the impact of respiration on both the arterial and venous pressure waveforms: respiration events impact vascular compliance of both arteries and veins, but because of the relatively low pressure within the veins, respiration has a more pronounced impact on the blood pressure therein.

When respiratory-induced modulation of both the arterial BP and PVP waveforms is removed, e.g., using a digital filtering technique, the agreement between the two signals is increased. For example, FIGS. 14A and 14B are graphs showing time-dependent plots of the beatpicks of these two waveforms (as opposed to the full-resolution waveforms that include every data point in addition to the beatpicks, as shown in FIGS. 11 and 12). FIG. 14A shows the arterial BP beatpicks, indicated by ‘o’ markers, while FIG. 14B shows the PVP beatpicks, indicated by ‘x’ markers. In all cases, the beatpicks where made using the IVDS beatpicking algorithm, as described above.

Both FIGS. 14A and 14B both include a dark, solid line indicating pressure variations wherein the respiratory artifact is digitally filtered out. Here, the filter used was a digital bandpass filter, with the limits of the filter consistent with the frequency at which respiration typically occurs (e.g., from about 3-20 breaths/minute). As is clear from the figure, the solid line generally passes through the respiratory-modulated beatpicks, and importantly illustrates the strong agreement in pressure variations for these signals when components related to respiration are removed.

In embodiments, the filter used to remove respiration components can be something other than a bandpass filter. Other candidate filters include a filter based on wavelets (e.g., CWT or DWT), an adaptive filter wherein respiration is measured with another technique (e.g., from the IMP waveform) and then used within a separate filter for PVP waveforms, a filter based in the frequency domain (e.g., one that is applied after the time-domain waveform is converted into a frequency-domain waveform using an FFT), or a simple smoothing algorithm. Other comparable digital filtering or digital signal-processing techniques for removing or reducing signal artifacts due to respiration modulation are within the scope of the invention.

Beatpicks from PVP waveforms correspond to systolic pressure within the vein, and typically have pressure values in the range of 10-30 mmHg, whereas those from arterial BP correspond directly to SYS and are relatively higher, e.g., typically in the range of 70-150 mmHg. Moreover, there does not appear to be universal relationship between venous and arterial pressures that applies to all patients. This means that, in order to estimate arterial BP from PVP waveforms, a calibration must be performed.

Referring to FIG. 15, a system for ‘calibrating’ a PVP waveform so that it can be used to estimate arterial BP values (SYS, MAP, and DIA) features the IVDS 80 according to the invention attached to an arm 23 of a patient 11, as described in detail with reference to FIG. 1. During the calibration period, which typically takes place at the beginning of a measurement, a blood pressure cuff 181 making an oscillometric measurement of BP attaches to the patient's brachial region (e.g., bicep). The blood pressure cuff 181 includes a flexible cuff 180 that wraps around the bicep; it features an inflatable bladder and is typically composed of a nylon-type material with Velcro® patches used to temporarily secure it. A control module 182 controls the blood pressure cuff 181 and features a circuit board containing a microprocessor, wireless Bluetooth® transceiver, pressure sensor, power circuitry, and analog/digital signal-conditioning electronics; an electronic pump; and a battery.

To initiate a measurement, a clinician (or the actual patient 11) presses an on/off button 184 on the blood pressure cuff 181. This activates the pump within the control module 182, causing it to inflate the bladder within the cuff, collect pressure signals from the patient's bicep, and generally perform a standard blood pressure measurement using oscillometry. This yields initial values of SYS, DIA, and MAP. Additionally, the pressure sensor within the blood pressure cuff 181 measures a time-dependent pressure waveform that indicates the pressure applied to the patient's brachial artery by the flexible cuff 180. Once measured, these parameters—values of SYS, DIA, and MAP, along with a time-dependent pressure waveform—are wirelessly transmitted by the Bluetooth® transceiver within the blood pressure cuff 181 to a paired Bluetooth® transceiver within the electronics module 94 enclosed by the arm-worn housing 20. More specifically, the microprocessor featured in the electronics module 94 receives and processes these parameters, along with other time-dependent waveforms measured by the IVDS 80, to determine a patient-specific calibration, as described in more detail below.

The Bluetooth® communication between the blood pressure cuff 181 and the electronics module 94 in the IVDS 80, as indicated by the arrow 188 in the figure, is a two-way connection: as described above, the blood pressure cuff 181 sends values of SYS, DIA, and MAP and a time-dependent pressure waveform to the IVDS 80, and this system processes this information to generate a patient-specific calibration, and can also send information (such as an acknowledgement, error code, or instruction to initiate a new calibration measurement) to the blood pressure cuff 181.

The patient-specific calibration is typically determined by collectively analyzing the time-dependent pressure waveform from the blood pressure cuff 181, along with time-dependent waveforms collected by the IVDS 80, e.g., IMP, temperature, PPG, and motion waveforms, and time-dependent PVP-AC and PVP-DC waveforms measured by the PVP-conditioning circuit board 95. Similar techniques have been described in the following U.S. Patents, the contents of which are incorporated herein by reference: Banet et al., Body-worn system for continuous, noninvasive measurement of cardiac output, stroke volume, cardiac power, and blood pressure, U.S. Pat. No. 10,722,131; Banet et al., Handheld physiological sensor, U.S. Pat. No. 10,206,600; McCombie et al., System for calibrating a PTT-based blood pressure measurement using arm height, U.S. Pat. No. 8,672,854; Banet et al., Cuffless system for measuring blood pressure, 7,179,228; and Banet et al., Blood-pressure monitoring device featuring a calibration-based analysis, 7,004,907.

More specifically, to determine the patient-specific calibration, Multiple values of PVP values and arterial BP values can be collected and analyzed to determine patient-specific slopes, which relate changes in PVP with changes in SYS, DIA, and MAP. The patient-specific slopes can also be determined using pre-determined values from a clinical study, and then combining these measurements with biometric parameters (e.g., age, gender, height, weight) collected during the clinical study. In still other embodiments, the patient-specific slope can be determined by detecting the change in PVP (as measured with the PVP-conditioning circuit board 95) with the change in applied pressure to the brachium (as measured with the control module 182 within the blood pressure cuff 181). Here, arterial pressure can be estimated from the variable pressure applied by the blood pressure cuff 181, and then correlated with the variably PVP measured during inflation of the cuff. This relationship can then be used to estimate the patient-specific calibration. Other calibration approaches, such as empirical methods based on the patient's biometric parameters, and as described in the above-mentioned patents, are also within the scope of the invention.

Once a measurement is complete, the IVDS 80 can wirelessly transmit numerical values through a Bluetooth® interface, as indicated by arrow 189, to an external display, such as an infusion pump 192. This type of communication, for example, allows for a closed-loop system wherein the infusion pump 192 delivers fluids to the patient to impact their BP, blood volume, and other physiological parameters, and the IVDS 80 determines whether or not the fluids are delivered to the patient's venous system or infiltrating into underlying tissue, and additionally how the patient is responding to the delivered fluids. In other embodiments, the IVDS 80 sends information through a similar wireless interface to another remote display, such as a mobile telephone, computer, tablet computer, television, hospital EMR, or another comparable display device.

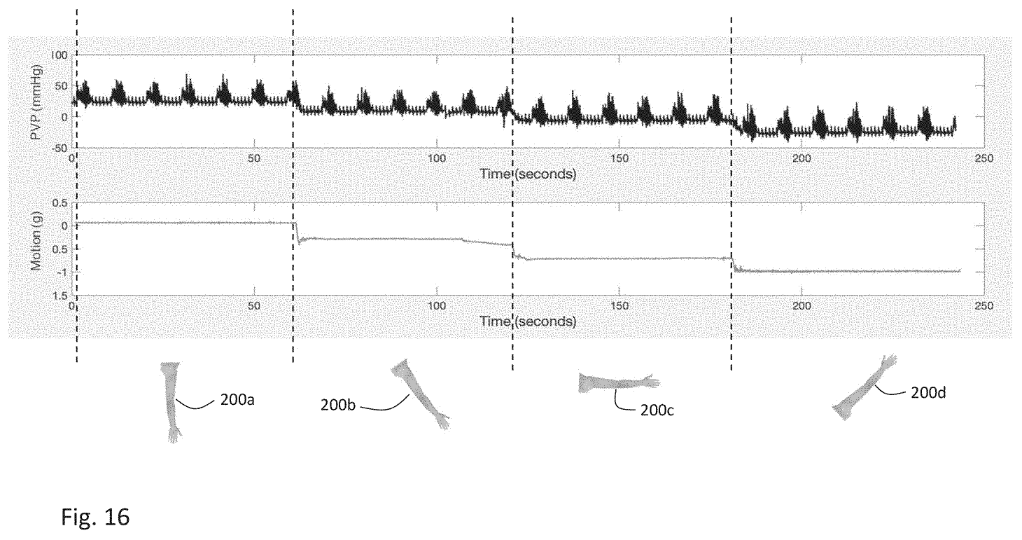

FIG. 16 shows how a patient's arm height can influence the PVP waveform, and in particular change both the baseline of the signal (which is readily apparent from the gross changes in FIG. 16) and the magnitude of each heartbeat-induced impedance pulse (a feature that is present upon close inspection of the data, but less apparent in FIG. 16). The graph in FIG. 16 shows time-dependent PVP and motion (taken from the accelerometers z-axis) waveforms measured at four different arm positions, as indicated by graphics 200a-d. During the first 60 seconds, the patient's arm is pointing directly downwards, as indicated by the graphic 200a, and the PVP waveform has an initial baseline of around 20 mmHg. For the next 60 seconds, the patient raises their arm by about 45° as indicated by the graphic 200b, causing the PVP waveform baseline to drop by about 20 mmHg. This trend continues as the patient raises their arm to 90° (as indicated by the graphic 200c), and finally to 135° (as indicated by the graphic 200d). FIG. 16 also shows how the accelerometer-measured motion signal (in this case, along the z-axis) changes with arm height in a commensurate way, thus indicating that this signal can be processed to estimate the actual arm height.

The change in PVP signals with arm height and the ability to automatically characterize the relative arm height with an accelerometer are important for several reasons. First, because both PVP and arterial BP change with a change in arm height in a continuous, well-defined manner, a process involving systematic variation of arm height may be used to calibrate a blood pressure measurement based on PVP, as described above. Second, because PVP signals (both baseline and heartbeat-induced pulses) vary with arm height, an accurate arterial BP measurement based on them will need to account for arm height, as measured with an accelerometer.

For the IVDS, calculating arm height from an accelerometer signal is preferably done by generating a series of look-up tables' beforehand that feature separate entries for both parameters, as characterized with a clinical trial involving subjects of varying demographics (e.g., height, weight, BMI, gender, age). The look-up tables are preferably coded into the IVDS's software during manufacturing. During an actual measurement, the accelerometer signals is measured and compared to the appropriate look-up table to estimate the arm height.

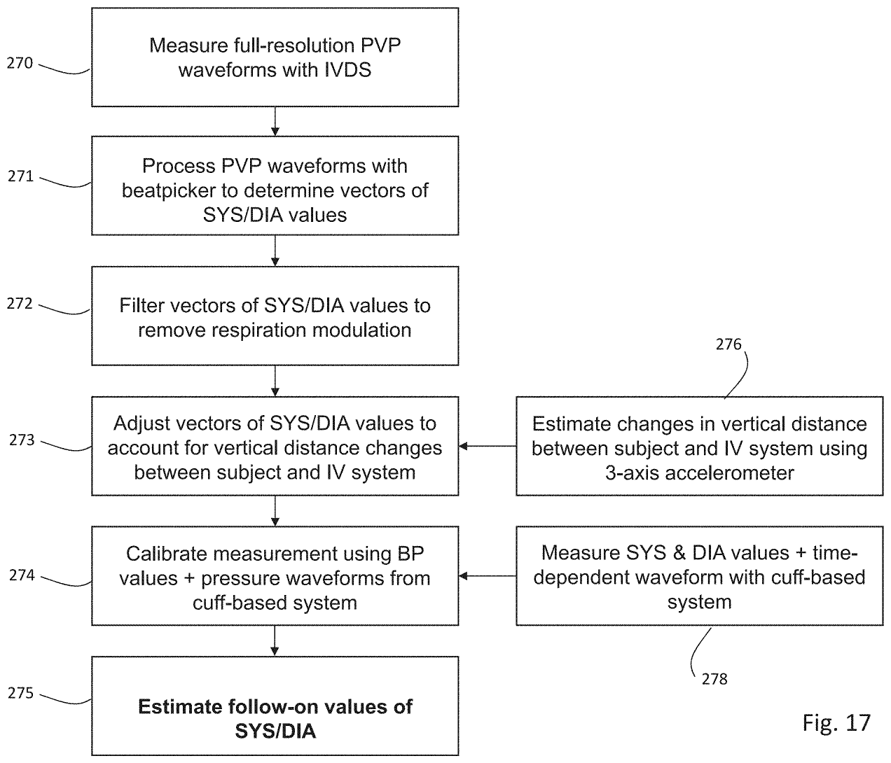

An algorithm based on the results shown in FIG. 14 (removal of respiration modulation using digital filtering), FIG. 15 (calibration with a cuff-based system), and FIG. 16 (accounting for arm height) can be used to estimate arterial BP from PVP. FIG. 17 shows a flow chart indicating the algorithm's primary steps. The algorithm begins (step 270) with measuring PVP waveforms using an IVDS like that shown in FIGS. 1 and 15. Such a system, for example, would be deployed on a hospitalized or surgical patient connected to a conventional IV system. After the IVDS measures PVP waveforms, it processes them with beatpicker, such as the IVDS beatpicking algorithm described above with reference to FIG. 10, to determine a collection of points (i.e., ‘vectors’) of SYS/DIA values (step 271). Using embedded computer code operating on the IVDS, the algorithm then filters vectors of SYS/DIA values to remove respiration modulation using one of the above-mentioned digital signal processing techniques, e.g., bandpass filter, adaptive filter, wavelet filter (e.g., CWT or DWT), simple multi-point smoothing function (step 272). Once filtered, the IVDS uses its internal multi-axis accelerometer to estimate changes in vertical distance between subject and IV system, as per the approach outlined with respect to FIG. 16 (step 276). The changes in vertical distance are then processed by the IVDS to adjust vectors of SYS/DIA values to account for vertical distance changes between the patient and IV system (step 273). When this is complete, the IVDS initiates a calibration measurement, as described above with reference to FIG. 15, wherein it instructs the blood pressure cuff to measure SYS & DIA values and a time-dependent pressure waveform (step 278). The algorithm uses these values from the cuff-based system to effectively calibrate the measurement, i.e., determine the initial values of SYS and DIA and to generate the patient-specific calibration (step 274). With this calibration and the PVP waveforms, the IVDS can estimate follow-on value of SYS/DIA (step 275).

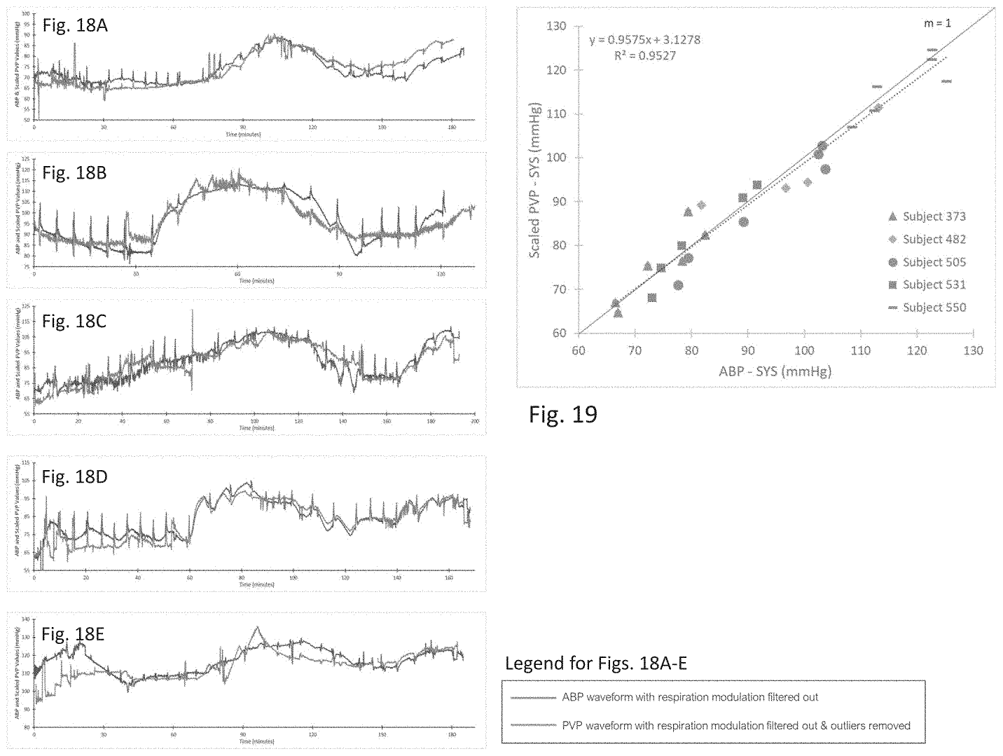

FIGS. 18 and 19 show the results of processing PVP data from five different porcine subjects using a version of the algorithm shown in FIG. 17. The plots in FIGS. 18A-E show time-dependent values of SYS taken from PVP (i.e., estimated SYS) and arterial BP waveforms (actual SYS). In each case, agreement between the estimated SYS and actual SYS is good, even during periods of blood pressure swings that are both large and rapid.

FIG. 19 shows a graph indicating the agreement between the estimated and actual SYS values, as taken from FIGS. 18A-E. Data points were selected every 30 minutes to generate this graph. From the pooled paired values used to generate the plot, the overall bias was calculated as 0.81 mmHg, and the standard deviation was 3.93 mmHg. The r-value indicating correlation was 0.98, indicating excellent agreement, and the slope of the data points was 0.96, indicating a near-unity value and general lack of any systematic variation. Taken collectively, these data indicate the efficacy of the blood pressure measurement described herein.

### 5. Measurement of Motion and Posture with the IVDS

The same accelerometer used in the IVDS to estimate arm height can also detect a patient's motion and posture, e.g., during a hospital stay. And importantly, it can be used to characterize periods of motion that may make the measurements described herein—IV infiltration and PVP-based BP—difficult or impossible because of motion-related artifacts. In short, the accelerometer can detect motion, which by itself is useful for characterizing a patient, while additionally indicating periods when the patient is relatively motion-free and a measurement can ideally be made.

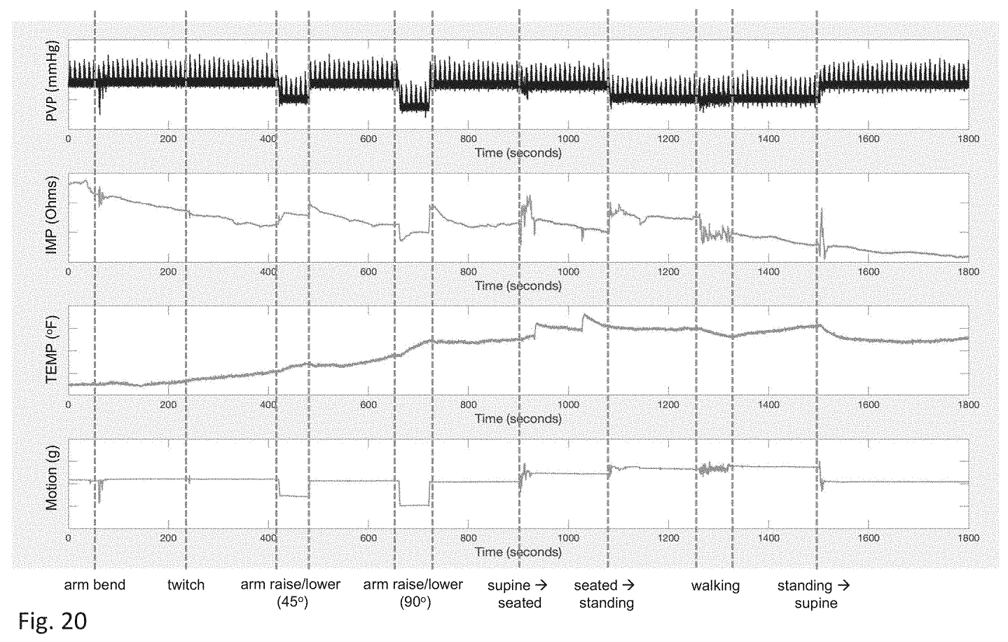

FIG. 20, for example, shows time-dependent PVP, IMP, temperature, and motion (from the z-axis of the accelerometer) waveforms measured during the following events: arm bends, twitching, arm raise and lower (45° and 90°), transitions from supine to seated and from seated to supine, walking, and the transition from standing to supine. Dashed lines in the figure delineate each event as a function of time. FIG. 19 indicates that each waveform is impacted by motion to some extent. The IMP waveform, in particular, is composed of relatively weak signals and is most profoundly impacted by motion; in particular activities that involved large arm movements, such as walking, impart large amounts of noise on the waveform.

In preferred embodiments, the microprocessor positioned on the IVDS's electronics module operates an algorithm that continuously processes signals from all 3 axes of the accelerometer. By comparing these data to that in a pre-determined look-up table, or alternatively first-principles models, the algorithm determines: 1) the type of motion the patient is undergoing; and 2) whether or not the motion is severe enough to impact the PVP-based blood pressure measurement, as well as measurements of other vital signs as described below. The IVDS reports a set of values when the motion is such that the algorithm determines that a measurement can be made.

In other embodiments, using information from the accelerometer, the IVDS can determine events that are about to occur, such as a patient moving around in a hospital bed and preparing to exit the bed. In these and other instances, the IVDS can wirelessly transmit an ‘alarm’ or an ‘alert’ to a remote display, e.g., an infusion pump as indicated in FIG. 15.

### 6. Measurement of Other Vital Signs and Physiological Parameters with the IVDS

The same sensors described herein that are used to detect IV infiltration—most notably the IMP, temperature, and the PVP-conditioning circuit board used to process PVP signals—can perform ‘double duty’ and additionally measure waveforms that yield other vital signs, such as HR, HRV, RR, and TEMP. Additionally, the IVDS can include a reflective optical system (typically disposed within the flexible, breathable polymeric base (component 89) in FIG. 1) that can be used to characterize IV infiltration using time-dependent changes in an optical signal. This same optical signal can simultaneously yield values of PR and SpO2. These measurements, when combined with the PVP-based BP measurement described herein, means the IVDS can potentially measure all five vital signs (HR, RR, TEMP, SpO2, and BP) typically used to characterize a patient.

Electrodes (i.e., components 83 in FIG. 1) sense signals that are used for the IVDS's bio-impedance (or, alternatively, bio-reactance) measurement, which yields a time-dependent IMP waveform that includes features related to HR and RR. Here, one pair of electrodes in the IVDS's polymeric base inject a high-frequency (e.g., 20-100 kHz), low-amplitude (e.g., 10-1000 □A) current into the patient's body. The current injected by the two electrodes is out of phase by 180°. The other pair of electrodes measure a voltage that, with follow-on processing, indicates the resistance (or impedance) encountered by the injected current. The voltage relates to the resistance (or impedance) through Ohms Law. Typically, a bio-impedance circuit within the electronic module measures IMP waveforms, which are separated into an AC waveform that features relatively high-frequency features (typically called □Z(t)), and a DC waveform that features relatively low-frequency features (typically called Z0). This technique for measuring □Z(t) and Z0 is described in detail in the following co-pending patent applications, the contents of which are incorporated herein by reference: “NECK-WORN PHYSIOLOGICAL MONITOR,” U.S. Ser. No. 62/049,279, filed Sep. 11, 2014; “NECKLACE-SHAPED PHYSIOLOGICAL MONITOR,” U.S. Ser. No. 14/184,616, filed Feb. 19, 2014; and “BODY-WORN SENSOR FOR CHARACTERIZING PATIENTS WITH HEART FAILURE,” U.S. Ser. No. 14/145,253, filed Dec. 31, 2013, and PHYSIOLOGICAL MONITORING SYSTEM FEATURING FLOORMAT AND WIRED HANDHELD SENSOR.

Physiological processes within a patient's arm modulate □Z(t) and Z0 waveforms sensed by the IVDS's bio-impedance measurement system. Thus processing these waveforms can yield parameters that correspond to the physiological processes. For example, respiratory effort (i.e., breathing), affect □Z(t) to impart a series of low-frequency undulations (typically 5-30 undulations/minute) on the waveform. The IVDS's electronics module processes these oscillations to determine RR. Blood is a good electrical conductor, and thus blood flow in the patient's arm manifests as heartbeat-induced cardiac pulses on the □Z(t) waveform. They can be processed with known techniques in the art to determine HR and HRV.

Physiological fluids in the arm also conduct the injected current. They can accumulate in this region (much like fluids accumulate to detect IV infiltration, albeit on a much slower time scale) and affect the impedance within the electrode's conduction pathway in a low-frequency (i.e., slowly changing) manner; processing the Z0 waveform can therefore detect them. Typically, the Z0 waveform features an average value of between about 10-50 Ohms, with 10 Ohms indicating relatively low impedance and thus high fluid content (e.g., the patient is ‘wet’), and 50 Ohms indicating a relatively high impedance and thus low fluid content (e.g., the patient is dry′). Time-dependent changes in the average value of Z0 can indicate that the patient's fluid level is either increasing or decreasing. An increase in fluid level, for example, may indicate the onset of congestive heart failure or kidney failure.

To measure optical signals, the IVDS may include a light source, e.g., a dual-emitting LED operating in a transmissive or reflective-mode geometry, which generates red and infrared optical wavelengths in the □=660 nm and □=908 nm region, and a photodetector (e.g., photodiode). These components measure PPG waveforms using both red and infrared radiation, as is generally known in the art, from either the patient's arm or one of their digits (e.g., the thumb) that is proximal to the IV site. The electronics module processes the waveforms to determine SpO2. Such measurement is described in more detail in the following co-pending patent applications, the contents of which are incorporated herein by reference: “NECK-WORN PHYSIOLOGICAL MONITOR”, U.S. Ser. No. 62/049,279, filed Sep. 11, 2014; “NECKLACE-SHAPED PHYSIOLOGICAL MONITOR”, U.S. Ser. No. 14/184,616, filed Feb. 19, 2014; and “BODY-WORN SENSOR FOR CHARACTERIZING PATIENTS WITH HEART FAILURE”, U.S. Ser. No. 14/145,253, filed Dec. 31, 2013. In general, and as explained in greater detail in these incorporated references, during an SpO2 measurement, the digital system alternately powers red and infrared LEDs within the dual-emitting LED. This process generates two distinct PPG waveforms. Using both digital and analog filters, the digital system extracts AC and DC components from the red (RED(AC) and RED(DC)) and infrared (IR(AC) and IR(DC)) PPG waveforms, which the digital system then processes to determine SpO2, as described in the above-referenced patent applications. To enhance the optical signal, the IVDS may include a thin film heating element, such as a Kapton® film with embedded electrical conductors arranged, e.g., in a serpentine pattern. Typically, the temperature of the heating element is regulated in a closed-loop manner at a level of between 41 to 42° C., which has minimal effect on the underlying tissue and is considered safe by the U.S. Food and Drug Administration (FDA).

Such an optical system and thin film heating element is described in the following patent application, the contents of which are incorporated herein by reference: “PATCH-BASED PHYSIOLOGICAL SENSOR” U.S. Ser. No. 16/044,386, filed Jul. 24, 2018.

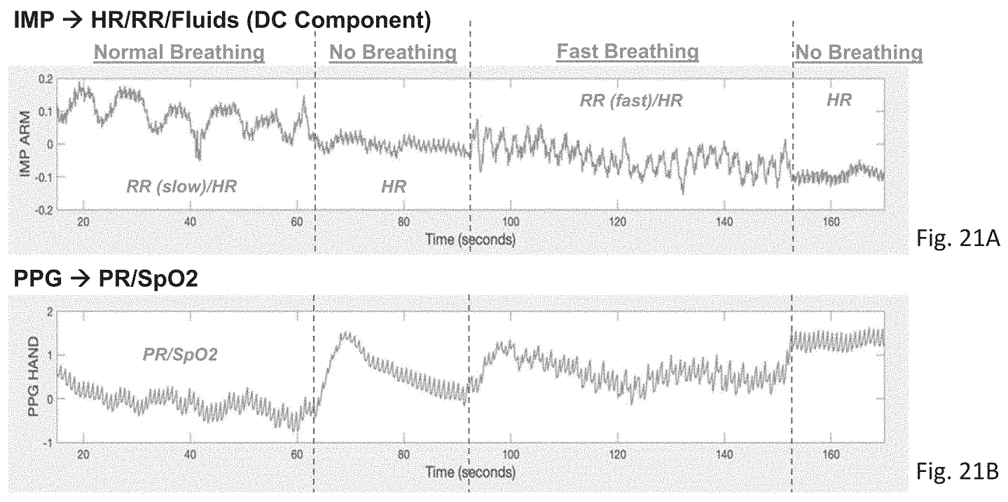

FIGS. 21A and 21B show graphs indicating IMP and PPG waveforms measured with a version of the IVDS shown in FIG. 1 from a subject participating in a clinical study. Similar results were obtained from 13 other subjects participating in the study. Here, the IVDS was applied to each subject's arm proximal to a conventional IV site. The subjects where then instructed to breathe at a normal rate, then hold their breath, then breathe at a fast rate, and then hold their breath once again. FIG. 21A shows an IMP waveform measured during this process. As is clear from the data, relatively small heartbeat-induced pulses are present throughout the measurement period. These are due to blood flow near the IV site. Additionally (and somewhat surprisingly), impedance signals measured from the arm were highly sensitive to respiration rate. From these data, along with those collected from other subjects, HR, HRV, and RR values could be calculated with reasonable accuracy. Importantly, the electrodes and circuit elements that are used for these measurements are the same as those used to detect IV infiltration, described in detail above.

Likewise, the optical sensor in the IVDS measured PPG waveforms using both RED an IR radiation. Typically, the waveform measured with IR radiation had a relatively high signal-to-noise ratio. From the PPG waveforms PR and SpO2 values were calculated, as described above. As with the above-described electrodes, the optical system used for these measurements is that same as that used to detect IV infiltration, as described above.

Additionally, the PVP waveform can be processed to determine HR, RR, and other hemodynamic parameters. These measurements can be used to offset or improve those made with IMP and PPG waveforms, as described with reference to FIG. 21. For example, calculating the FFT of the PVP waveform yields a frequency-domain spectrum featuring peaks that correspond to HR (F1) and RR (F0). Features associated with F0 and F1 (e.g., their amplitude or energy) may be processed in different ways to estimate fluid-related parameters, e.g., wedge pressure and/or pulmonary arterial pressure. Further processing of the energy then yields the appropriate fluid-related parameters. Examples of such processing are described in the following references, the contents of which have been already incorporated herein by reference:
1) Hocking et al., “Peripheral venous waveform analysis for detecting hemorrhage and iatrogenic volume overload in a porcine model.”, Shock. 2016 October; 46(4):447-52;
2) Sileshi et al., “Peripheral venous waveform analysis for detecting early hemorrhage: a pilot study.”, Intensive Care Med. 2015 June; 41(6): 1147-8;
3) Miles et al., “Peripheral intravenous volume analysis (PIVA) for quantitating volume overload in patients hospitalized with acute decompensated heart failure—a pilot study.”, J Card Fail. 2018 August; 24(8):525-532; and
4) Hocking et al., “Peripheral i.v. analysis (PIVA) of venous waveforms for volume assessment in patients undergoing haemodialysis.”, Br J Anaesth. 2017 Dec. 1; 119(6):1135-1140.

In other embodiments, the IVDS may collectively process hemodynamic parameters measured PVP waveform (e.g., wedge pressure and blood volume, which may be correlates with energies associated with F0, F1, or some combination thereof) with those measured by other sensors within the IVDS (e.g., BP, SpO2) to determine the patient's fluid status and effectively inform delivery of fluids while resuscitating the patient (e.g., during periods of sepsis and/or fluid overload). In general, by using information from both the PVP waveform and IVDS, a clinician can better manage the patient 11 by characterizing life-threatening conditions and help guide their resuscitation.

As a more specific example, in embodiments values of BP and SpO2 measured by the IVDS can be combined with volume status determined from the PVP waveform to estimate a patient's blood flow and perfusion. Knowledge of these parameters, in turn, can inform estimation of how much fluid a clinician needs to deliver upon resuscitation. Similarly, BP, and SpO2 measured by the IVDS, along with the ratio of F0 and F1 energies measured from the PVP waveform, each indicate a patient's level of perfusion. They can also be combined in a mathematical ‘index’ to better estimate this condition. Then these parameters or the index can be measured while the patient undergoes a technique called a ‘passive leg raise’, which is a test to evaluate the need for further fluid resuscitation in a critically ill person. The passive leg raise involves raising a patient's legs (typically without their active participation), which causes gravity to pull blood from the legs into the central organs, thereby increasing circulatory volume available to the heart (typically called ‘cardiac preload’) by around 150-300 milliliters, depending on the amount of venous reservoir. If the above-mentioned parameters or index measured by the IVDS increase, this can indicate that the leg raise effectively increases perfusion in the patient's central organs, thereby indicating that they will be responsive to fluids. Clinicians can perform a similar test by providing the patient a bolus of fluids through an IV system, and then monitoring the increase or decrease in the parameters or index measured by the IVDS.

In embodiments, simple linear computational methods, combined with results from clinical studies, can be used to develop models that collectively process data generated by the IVDS. In other embodiments, more sophisticated computational models, such as those involving artificial intelligence and/or machine learning, can be used for the collective processing.

### 7. Other Embodiments

In other embodiments, time and frequency-domain analyses of IMP, PPG, PVP, and motion waveforms can be used to distinguish respiratory events such as coughing, wheezing, and to measure respiratory tidal volumes. In particular, respiratory tidal volumes are determined by integrating the area underneath a ‘respiratory pulse’ in an IMP or BR waveform (such as that indicated in FIG. 21A), and then comparing this to a pre-determined calibration. Such events may be combined with information from the IVDS to help predict patient decompensation. In other embodiments, the IVDS may use variations of the algorithms described above for determining vital signs and hemodynamic parameters. For example, to improve the signal-to-noise ratio of pulses within the IMP and PPG waveforms, embedded firmware operating on the patch sensor can operate a signal-processing technique called ‘beatstacking’. With beatstacking, for example, an average pulse is calculated from multiple (e.g., seven) consecutive pulses from the IMP waveform, which are delineated, and then averaged together. The derivative of the AC component of the IMP waveform is then calculated over a 7-sample window as an ensemble average, and then used as described above.

Other embodiments are within the scope of the invention. For example, other components of signals measured with the sensors within the IVDS, and particularly those used to measure PVP waveforms, can be analyzed to evaluate the patient.

In embodiments, for example, the arterial pulse pressure (herein “PP”) can be calculated from SYS and DIA as described above, and then analyzed to estimate a change in the patient's volume status, as less blood volume can lower arterial pulse pressure and more blood volume can raise arterial pulse pressure. Additionally, the venous system stores 60-70% of the blood volume and serves as a volume reservoir, and is a highly compliant, low-pressure system that can accommodate large changes in volume with minimal changes in pressure. The amplitude and shape of the PVP waveform has been demonstrated to be sensitive to changes in intravascular volume in recent studies. Changes in intravascular volume status in both humans and pigs led to changes in the PVP waveform before changes in arterial BP, HR, and the pulmonary artery diastolic pressure, suggesting that the PVP waveform is more sensitive to changes in intravascular volume than standard vital signs.

A venous segment's PVP waveform during a given cardiac cycle is the direct result of the blood volume changes that occur within that vein segment and the vein segment's compliance. The vein segment's compliance is expected to be constant during a given cardiac cycle and the corresponding compliance values over the duration of the cardiac cycle are determined by blood inflow and outflow for a given vein segment. Thus, the change in a vein segment's PVP during a given cardiac cycle is the result of the change in blood volume within the vein segment that occurs during a given cardiac cycle (i.e., the net effect on volume change resulting from blood flowing into and out of the vein segment). Based on the anatomical considerations and the results of the cited studies based on physiologic models, changes in PVP waveforms detected in a peripheral vein segment are due to net changes in the segment's blood volume over the course of each cardiac cycle.

Since the cyclical blood volume change (and corresponding cyclical pressure change) in a vein segment results from cardiac-induced cyclical change in flow into, and out of, the vein segment, the blood volume change in a vein segment results from the interaction of inflow pressure, outflow pressure, and intraluminal pressure. Thus, analysis of these parameters from the PVP waveform, as measured with the IVDS, may yield information concerning a patient's hemodynamic state.

When downstream resistance to venous return increases (for example, during atrial contraction or when the tricuspid valve closes), outflow pressure will increase. This causes a reduction (and eventual cessation once the proximal vein segment valve closes) of blood flow out of a given vein segment into the adjacent, downstream vein segment. Simultaneous, blood flow from the adjacent, upstream segment into the vein segment will continue but also decrease (and eventual cessation once the distal vein segment valve closes). The net effect of these two actions will increase the blood volume within the vein segment (where the PVP sensor is located) distending its walls outward and increasing intraluminal pressure (corresponding to the upstroke of the PVP waveform). Peak intraluminal pressure within the vein segment will occur just prior to the point when that pressure becomes greater than the outflow pressure.

In contrast, when downstream resistance to venous return decreases (for example, during atrial relaxation or when the tricuspid valve opens), outflow pressure will decrease. This causes an increase (and eventual cessation once the proximal vein segment valve closes) in blood flow out of a given vein segment into the adjacent, downstream vein segment. Simultaneous, blood flow out of the adjacent, upstream segment into the vein segment will begin to increase (and eventual cessation once the distal vein segment valve closes). The net effect of these two actions will decrease the blood volume within the vein segment (where the PVP sensor is located) allowing its walls to recoil and intraluminal pressure to decrease (corresponding to the downstroke of the PVP waveform). The vein segment intraluminal pressure nadir will occur just prior to the point when intraluminal pressure becomes less than the outflow pressure.

In summary, the PVP waveform measured from a vein segment is highly dependent on: i) the cycle of the right heart altering atrial volume and hence, atrial pressure, which in turn dictates venous return (i.e., venous outflow for a given peripheral vein segment; ii) blood flow out of the adjacent upstream vein segment into the adjacent downstream vein segment (i.e., venous inflow for a given peripheral vein segment); and iii) the compliance of the venous wall in that vein segment, which can be affected by changes in venous tone. All combined define the amplitude and shape of the PVP waveform.

Hypovolemia (e.g., blood loss, dehydration) has been shown to reduce the amplitude of PVP waveforms. Potential mechanisms for these findings include low arterial blood flow and blood pressure feeding the capillaries may lead to lower venous inflow and pressure, causing slower and/or reduced venous filling causing a more gradual upslope and/or lower peak venous pressure. Initially, hypovolemia may lower venous inflow (upstream) pressure more than venous outflow (downstream) pressure. This may lead to a more gradual downslope of the PVP waveform due to a reduced pressure gradient for blood flow out of the vein segment. Vasoconstriction in response to hypovolemia might exacerbate this effect if the vasoconstriction affects the arteries more than veins.

Lower venous inflow (upstream) pressure may also lead to a more gradual upslope of the PVP if the slower rate of venous filling does not allow the segment to reach maximum potential intraluminal pressure/distension before the right atrium either relaxes or the tricuspid valve opens allowing the downstream veins to start emptying.

As blood flows from the peripheral venous compartment to the central venous compartment falls, reduced downstream venous pressures can lower outflow pressure so that the maximum pressure change that can be achieved in the peripheral venous segment is reduced.

Even without changing the absolute blood volume, decreasing vasomotor tone simulates hypovolemia with some hemodynamic changes similar to those of absolute hypovolemia (e.g., reduced central pressures by reducing the stressed circulatory volume that generates venous return, reduced mean arterial pressure and potentially reduced cardiac output that can lead to reduced venous inflow pressure, and reduced venous intraluminal pressure). Lower venous tone also may lead to a more gradual upstroke and downstroke of the PVP waveform as more volume is required to increase the pressure in the vein segment when vessel diameter is increased. Similarly, increased venous tone can lead to the opposite effects—a steeper upstroke and downstroke of the vein segment PVP waveform.

In summary, PVP waveform's amplitude and shape primarily reflect changes in volume of the vein segment (where the PVP sensor is located) resulting from the interaction of blood inflow and blood outflow as the result of the changes in downstream or central venous volume/pressure changes driven by the cyclical contraction-relaxation of the right heart. The measured PVP waveform likely reflects the effective intravascular volume (the “stressed volume”, or the volume contributing to venous return and cardiac output) more closely than the absolute blood volume.

Other embodiments are within the scope of the invention. For example, signal-processing techniques outside (or in addition to) those described above can process PVP waveforms to isolate and improve the signal-to-noise ratio of PVP-AC and PVP-DC signal components, and particularly PVP-AC components. One such signal-processing technique is referred to as ‘wavelet decomposition’ and relates to the above-mentioned technique based on wavelet transforms. Wavelet decomposition algorithms approximate the PVP-AC signal with a collection of ‘wavelets’, each occurring at a different frequency (and usually octaves of each other). The algorithm only selects wavelets of certain, well-defined frequencies that are theoretically present in the desired signal, and then recombines these to approximate the PVP-AC signal. Wavelet decomposition can often yield reconstructed PVP-AC signals that indicate cardiac and respiratory pulses in a manner that is superior to conventional signal-processing techniques, such as infinite impulse response (herein ‘IIR’) filters commonly used in band-pass and low-pass filters. Additionally, wavelet decomposition is typically particularly effective in isolating PVP-AC pulses when pressure fluctuations due to pump activity, i.e. ‘pump noise’, is present and features similar frequency components compared to the PVP-AC signals.

In other embodiments, aimed at further increasing the signal-to-noise ratio of the PVP-AC signals, the tubing used to couple the venous catheter to the pressure transducer may be optimized. For example, the durometer (e.g., stiffness) of typically medical-grade tubing used in venous catheters is about 50-55 Shore A. Increasing this by roughly 25%, so that it is consistent with the durometer of tubing used for conventional arterial lines, increases the conductivity of high-frequency PVP-AC pulses so that they effectively and propagate in the tubing with minimal loss and are more readily detected. In related embodiments, the ‘fluid column’ within the tubing may be pressurized (e.g., using an external, pressurized IV bag filled with saline that is connected to the tubing), to further increase the tube's conductivity of the PVP-AC signals.

One purpose of analyzing PVP signals is to estimate a patient's volume status, and more specifically how the patient will respond to fluids. More specifically, it may be useful to determine where the patients ‘falls’ on the Frank-Starling curve, which plots stroke volume (e.g., flow) vs. pre-load (e.g., blood volume). A patient that is relatively ‘low’ on the curve will likely respond favorably to fluids, meaning their stroke volume may increase with increasing volume, which in turn is facilitated by increasing fluids. Conversely, a patient that is relatively ‘high’ on the curve may show little increase in flow when volume is increased. As such, an increased volume may drive the patient into a deleterious congestive state, such as congestive heart failure.

To this end, analysis of PVP-AC signals may yield a metric indicating how responsive the patient will be to infused fluids. This may include, for example, analysis of cardiac and respiratory components from the PVP-AC signals—wherein the signals are first processed using wavelet decomposition as described above—and then processing the resultant signals with an approach based on FFT or IIR filters to evaluate the relative magnitudes of both cardiac and respiratory components. Typically, for example, a patient will be responsive to fluids (e.g., their SV will subsequently increase) when the magnitude of the cardiac component is relatively small compared to the respiratory component. By using such data (typically collected during a clinical study) an embodiment of the invention may feature a simple ‘index’ that indicates the patient's responsivity to fluids. Such an index, for example, may be numerical (e.g., on a scale from 1-10), colorimetric (e.g., using ‘red’ to denote a patient in need of fluids; ‘green’ to denote a patient that is not in need of fluids), or something equivalent.

In still other embodiments, the index or other suitable metric for estimating the patient's fluid volume and/or responsivity may be based on the mean value of the PVP signal (herein “PVP-mean”), which is comparable to PVP-DC. PVP-mean indicates the mean pressure of the PVP signal. It has the advantage of always being present from the patient and relatively easy to process, mostly because it lacks oscillatory components related to the patient's cardiac or respiratory actions. Clinical work with the systems described herein indicates that that PVP-mean tracks a patient's receptivity to fluids when evaluated, for example, with lower body negative pressure (herein “LBNP”) clinical protocols. LBNP is an experimental maneuver that serves as a surrogate for hemorrhage—during LBNP, a subject's lower extremities are exposed to a systematically changing vacuum. This process pulls fluids from the subject's torso in a manner similar to hemorrhage. When the vacuum is released, blood and other fluids rush back into the subject's torso; this is analogous to transfusing blood back to a patient. Using the systems described herein, a surprising result of LBNP maneuvers applied to healthy subjects was that PVP-mean, along with the cardiac component of PVP-AC, systematically increased with increasing LBNP vacuum, and then rapidly returned to normal values once the vacuum was released. Thus, an index that includes PVP-mean by itself, or alternatively combined with components extracted from PVP-AC, can be used according to the invention to provide an index that indicates the patient's responsivity to fluids.

In yet another aspect of the invention, a ‘signal quality index’ (herein “SQI”) may be used with the above-described parameters (e.g. PVP-AC and the signal components therein; PVP-mean) to generate a comparable index. SQI is a metric that typically indicates the prevalence of a cardiac component in the PVP-AC signal: a low SQI indicates low amounts of a cardiac component, whereas a high SQI indicates high amounts of a cardiac component. Thus, low SQI values typically indicate a patient in need of fluids, whereas high SQI values typically indicate a patient with adequate fluids.

In still other embodiments of the invention, the PVP-monitoring components described herein may be coupled to other patient-worn sensors. For example, the patient may include a dressing or adhesive wrap that holds the venous catheter in place and simultaneously monitors the degree to which fluids or medication delivered by the IV ‘infiltrate’ out of the vein and into the 3rd space near the venous punction site. Signals measured by the dressing may be used to better process PVP-AC signals, as described herein. Conversely, the presence of PVP-AC signals indicate that a venous catheter is indeed properly in a patient's vein, and thus may be used with signals generated by the dressing to determine if fluids and/or medication delivered to the patient are infiltrating into their 3rd space.

These and other embodiments of the invention are deemed to be within the scope of the following claims.

What is claimed is:

1. A system for determining an arterial blood pressure value from a patient, comprising:
a catheter configured to insert into the patient's venous system;
a pressure sensor connected to the catheter and configured to measure physiological signals indicating a pressure in the patient's venous system; and
a processing system configured to: i) receive the physiological signals from the pressure sensor; and ii) process the physiological signals with an algorithm to determine the arterial blood pressure value.

2. The system of claim 1, wherein the processing system is further configured to operate an algorithm that filters out respiratory components from the physiological signals to determine the arterial blood pressure value.

3. The system of claim 2, wherein the algorithm is further configured to operate a bandpass filter to filter out respiratory components from the physiological signals.

4. The system of claim 2, wherein the algorithm is further configured to operate a filter based on wavelets to filter out respiratory components from the physiological signals.

5. The system of claim 1, wherein the processing system is enclosed by an enclosure that is configured to attach directly to the patient.

6. The system of claim 1, wherein the processing system further comprises a motion-detecting sensor.

7. The system of claim 6, wherein the motion-detecting sensor is one of an accelerometer and a gyroscope.

8. The system of claim 6, wherein the processing system is further configured to receive signals from the motion-detecting sensor and process them to determine the patient's degree of motion.

9. The system of claim 8, wherein the processing system is further configured to collectively process the patient's degree of motion and the physiological signals to determine the arterial blood pressure value.

10. The system of claim 6, wherein the processing system is further configured to receive signals from the motion-detecting sensor and process them to determine a relative height associated with a body part associated with the patient.

11. The system of claim 10, wherein the body part is the patient's arm.

12. The system of claim 10, wherein the processing system is further configured to collectively process the relative height associated with the body part associated with the patient and the physiological signals to determine the arterial blood pressure value.

13. The system of claim 1, wherein the processing system is further configured to receive a calibration blood pressure value from an external source.

14. The system of claim 13, wherein the processing system is further configured to process the calibration blood pressure value with the physiological signals to determine the arterial blood pressure value.

15. The system of claim 14, wherein the external source is one of a blood pressure cuff and an arterial catheter.

16. The system of claim 14, wherein the processing system is further configured to process a patient-specific relationship between venous blood pressure and arterial blood pressure, along with the calibration blood pressure value and the physiological signals, to determine the arterial blood pressure value.

17. The system of claim 16, wherein the processing system is further configured to process the physiological signals to determine the patient-specific relationship between venous blood pressure and arterial blood pressure.

18. The system of claim 16, wherein the processing system is further configured to process biometric information corresponding to the patient to determine the patient-specific relationship between venous blood pressure and arterial blood pressure.

19. A system for determining an arterial blood pressure value from a patient, comprising:
a catheter configured to insert into the patient's venous system;
a pressure sensor connected to the catheter and configured to measure physiological signals indicating a pressure in the patient's venous system;
a motion sensor configured to measure motion signals; and,
a processing system configured to: i) receive the physiological signals from the pressure sensor; ii) receive the motion signals from the motion sensor; iii) process the motion signals by comparing them to a pre-determined threshold value to determine when the patient has a relatively low degree of motion; and iv) process the physiological signals to determine the arterial blood pressure value.

20. A system for determining an arterial blood pressure value from a patient, comprising:
a catheter configured to insert into the patient's venous system;
a pressure sensor connected to the catheter and configured to measure physiological signals indicating a pressure in the patient's venous system;
a motion sensor configured to measure motion signals; and,
a processing system configured to: i) receive the physiological signals from the pressure sensor; ii) receive the motion signals from the motion sensor; iii) process the motion signals to determine a relative height between a body part associated with the patient and an infusion system; and iv) process the physiological signals and the relative height to determine the arterial blood pressure value.
